(19) United States
(12) Patent Application Publication
(10) Pub. No.: US 2011/0059940 A1
(43) Pub. Date: Mar. 10, 2011

(54) 2-Aryl Glycinamide Derivatives

(75) Inventors: Paul J. Gilligan, Wilmington, DE (US); Michael G. Yang, Narbeth, PA (US); Jianliang Shi, Madison, CT (US)

(73) Assignee: Bristol- Myers Squibb Company

(21) Appl. No.: 12/990,922

(22) PCT Filed: May 7, 2009
(86) PCT No.: PCT/US09/43116

§ 371 Date: Nov. 3, 2010

Related U.S. Application Data
(60) Provisional application No. 61/051,413, filed on May 8, 2008.

(51) Int. Cl. A61K 31/18; C07C 311/00; C07D 205/04; C07D 233/61; C07D 249/08; C07D 231/12; C07D 213/42; C07D 217/24; A61K 31/24; A61K 31/195; A61K 31/397; A61K 31/4164; A61K 31/4196; A61K 31/415; A61P 25/00; A61P 25/28

(52) U.S. Cl. 514/210.17; 560/13; 562/430; 564/92; 548/953; 548/343.5; 548/269.4; 548/377.1; 546/337; 546/141; 514/539; 514/562; 514/604; 514/399; 514/383; 514/406

(57) ABSTRACT

The disclosure provides compounds of Formula I, including pharmaceutically acceptable salts, their pharmaceutical compositions, and their uses in inhibiting β-amyloid peptide (β-AP) production.

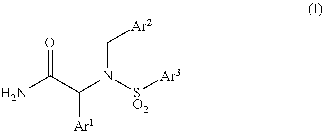

### CROSS-REFERENCE TO RELATED APPLICATIONS

This application claims the benefit of U.S. provisional application Ser. No. 61/051,413 filed May 8, 2008.

### BACKGROUND OF THE INVENTION

The disclosure provides compounds of Formula I, including pharmaceutically acceptable salts, their pharmaceutical compositions, and their uses in inhibiting β-amyloid peptide (β-AP) production.

Alzheimer's Disease is a progressive, neurodegenerative disorder characterized by memory impairment and cognitive dysfunction. Alzheimer's Disease is characterized pathologically by the accumulation of senile (neuritic) plaques, neurofibrillary tangles, amyloid deposition in neural tissues and vessels, synaptic loss, and neuronal death. It is the most common form of dementia and it now represents the third leading cause of death after cardiovascular disorders and cancer. The cost of Alzheimer's Disease is enormous (greater than $100 billion annually in the U.S.) and includes the suffering of the patients, the suffering of families, and the lost productivity of patients and caregivers. As the longevity of society increases, the occurrence of Alzheimer's disease will markedly increase. It is estimated that more than 10 million Americans will suffer from Alzheimer's disease by the year 2020, if methods for prevention and treatment are not found. Currently, Alzheimer's disease is estimated to afflict 10% of the population over age 65 and up to 50% of those over the age of 85. There is currently no effective treatment.

There have been many theories relating to the etiology and pathogenesis of Alzheimer's disease. These theories were either based on analogies with other diseases and conditions (e.g., slow virus and aluminum theories), or based on pathologic observations (e.g., cholinergic, amyloid, or tangle theories). Genetic analysis can potentially differentiate between competing theories. The identification of mutations in the β-amyloid precursor protein (β-APP) of individuals prone to early onset forms of Alzheimer's disease and related disorders strongly supports the amyloidogenic theories.

The β-amyloid precursor protein (β-APP), a large membrane spanning glycoprotein found in tissues of mammals, including humans, is encoded by a gene on the long arm of human chromosome 21. The main constituent of the plaques, tangles and amyloid deposits is known to be β-amyloid peptides (β-AP), composed of approximately 39 to 43 amino acid fragments of β-APP, and in particular, the 40 amino acid fragment known as Aβ1-40. Several lines of evidence support the involvement of β-AP in the pathogenesis of Alzheimer's disease lesions. β-AP and related fragments have been shown to be toxic for PC-12 cell lines and primary cultures of neurons, as well as causing neuronal degeneration with accompanying amnesia in rodents. Strong evidence for the role of β-AP in Alzheimer's disease consists of observations of genetic β-APP mutations in individuals with certain forms of Familial Alzheimer's Disease (FAD) and the correlation of disease onset with altered release of β-AP fragments.

It is presently believed that the development of amyloid plaques in the brains of Alzheimer's disease patients is a result of excess production and/or reduced clearance or removal of β-AP. It is known that a basal level of β-AP production may be a normal process and that multiple pathways for cleavage of β-APP exist. Currently, however, it is unclear which classes of proteinases or inhibitors thereof that would be effective in treating Alzheimer's disease. Various peptidergic compounds and their pharmaceutical compositions have been disclosed as useful in inhibiting or preventing amyloid protein deposits in brains of Alzheimer's disease and Down's Syndrome patients.

Thus, there is a clear need to develop compounds effective against β-amaloid production or accumulation. The invention provides technical advantages, for example, the compounds are novel and are effective against hepatitis C. Additionally, the compounds provide advantages for pharmaceutical uses, for example, with regard to one or more of their mechanism of action, binding, inhibition efficacy, target selectivity, solubility, safety profiles, or bioavailability.

N-benzenesulfonamido-1-(substituted)glycineamides have been disclosed. See Parker, M. F. et al., PCT application WO 03/053912, published Jul. 3, 2003.

### DESCRIPTION OF THE INVENTION

The invention encompasses compounds of Formula I, including pharmaceutically acceptable salts and solvates, their pharmaceutical compositions, and their uses in inhibiting β-amyloid peptide (β-AP) production.

One aspect of the invention are compounds of Formula I

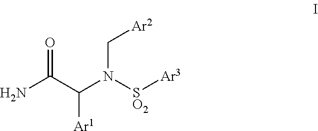

wherein:
Ar¹ is phenyl substituted with 0-5 substituents selected from the group consisting of halo, trifluoromethyl, cyano, C₁₋₆alkyl, and C₁₋₆alkoxy;
Ar² is phenyl or pyridinyl substituted with 0-5 substituents selected from the group consisting of halo, trifluoromethyl, cyano, C₁₋₆alkyl, C₁₋₆alkoxy, CO₂R¹, CON(R¹)(R¹), CON(R²)(R³), and Ar⁴,
or is

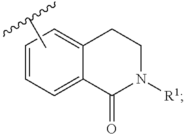

### Ar³ is

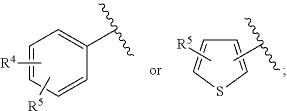

Ar⁴ is a heteroaryl moiety selected from the group consisting of imidazolyl, pyrazolyl, oxadiazolyl, oxazolyl, and triazolyl and is substituted with 0-2 C₁₋₆alkyl;
R¹ is independently hydrogen, C₁₋₆alkyl, C₃₋₇cycloalkyl, or (C₁₋₄alkoxy)C₁₋₄alkyl;
R² and R³ taken together are CH₂CH₂CH₂, CH₂CH₂CH₂CH₂, CH₂CH₂CH₂CH₂CH₂, CH₂CH₂CH(OH)CH₂CH₂, CH₂CH₂OCH₂CH₂, CH₂CH₂SCH₂CH₂, or CH₂CH₂N(CH₃)CH₂CH₂.
R⁴ is halogen; and
R⁵ is hydrogen or halogen;
or a pharmaceutically acceptable salt or solvate thereof.

Another aspect of the invention is a compound of formula I where

Ar¹ is phenyl, dihalophenyl, alkylphenyl, haloalkylphenyl, or alkoxyphenyl;
Ar² is phenyl substituted with 1 substituent selected from the group consisting of halo, trifluoromethyl, cyano, CO₂R¹, CON(R¹)(R¹), CON(R²)(R³), and Ar⁴;
or Ar² is pyridinyl or

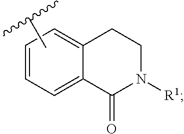

Ar³ is halophenyl;
Ar⁴ is imidazolyl, pyrazolyl, oxazolyl, triazolyl, or oxadiazolyl, and is substituted with 0-1 C₁₋₆alkyl;
R¹ is independently hydrogen, C₁₋₆alkyl, or C₃₋₇cycloalkyl; and
R² and R³ taken together is CH₂CH₂CH₂;
or a pharmaceutically acceptable salt thereof.

Another aspect of the invention is a compound of formula I where

Ar¹ is phenyl, difluorophenyl methylphenyl, trifluoromethylphenyl, or methoxyphenyl;
Ar² is fluorophenyl, trifluoromethylphenyl, cyanophenyl, (alkoxycarbonyl)phenyl, (carboxy)phenyl, (N-methylaminocarbonyl)phenyl, (N-ethylaminocarbonyl)phenyl, (N-t-butylaminocarbonyl)phenyl, (cyclobutylaminocarbonyl)phenyl, (N,N-dimethylaminocarbonyl)phenyl, (azetdinylcarbonyl)phenyl, (pyrazolyl)phenyl, (imidazolyl)phenyl, (triazolyl)phenyl, (oxazolyl)phenyl, (oxadiazolyl)phenyl, (methyloxadiazolyl)phenyl, pyridinyl, or (N-ethyloxotetrahydroisoquinolinyl; and
Ar³ is chlorophenyl;
or a pharmaceutically acceptable salt thereof.

Another aspect of the invention are compounds of Formula I where Ar¹ is phenyl substituted with 0-3 substituents selected from the group consisting of halo, trifluoromethyl, cyano, C₁₋₆alkyl, and C₁₋₆alkoxy.

Another aspect of the invention are compounds of Formula I where Ar¹ is phenyl substituted with 1-2 substituents selected from the group consisting of halo, trifluoromethyl, cyano, C₁₋₆alkyl, and C₁₋₆alkoxy.

Another aspect of the invention are compounds of Formula I where Ar¹ is phenyl, halophenyl, dihalophenyl, methylphenyl, trifluoromethylphenyl, or methoxyphenyl and where halo is chloro or fluoro.

Another aspect of the invention are compounds of Formula I where Ar² is phenyl substituted with 0-3 substituents selected from the group consisting of halo, trifluoromethyl, cyano, C₁₋₆alkyl, C₁₋₆alkoxy, CO₂R¹, CON(R¹)(R¹), CON(R²)(R³), and Ar⁴.

Another aspect of the invention are compounds of Formula I where Ar² is phenyl substituted with 1-2 substituents selected from the group consisting of halo, trifluoromethyl, cyano, C₁₋₆alkyl, C₁₋₆alkoxy, CO₂R¹, CON(R¹)(R¹), CON(R²)(R³), and Ar⁴.

Another aspect of the invention are compounds of Formula I where Ar² is phenyl substituted with 1 substituent selected from the group consisting of cyano, CO₂R¹, CON(R¹)(R¹), and CON(R²)(R³).

Another aspect of the invention are compounds of Formula I where Ar² is phenyl substituted with 1 Ar⁴.

Another aspect of the invention are compounds of Formula I where Ar² is phenyl substituted with 1 substituent in the para position.

Another aspect of the invention are compounds of Formula I where Ar² is

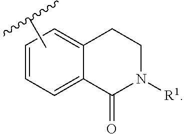

Another aspect of the invention are compounds of Formula I where Ar² is

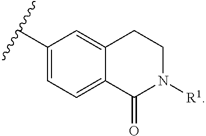

Another aspect of the invention are compounds of Formula I where Ar³ is 4-chlorophenyl.

Another aspect of the invention are compounds of Formula I where Ar⁴ is imidazolyl, pyrazolyl, oxazolyl, oxadiazolyl, triazolyl, methylimidazolyl, methylpyrazolyl, methyloxadiazolyl, or methyltriazolyl.

Another aspect of the invention are compounds of Formula Ia.

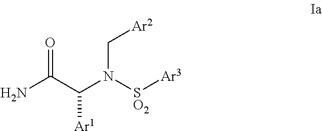

For a compound of Formula I, the scope of any instance of a variable substituent, including R¹, R², R³, R⁴, R⁵, Ar¹, Ar², Ar³, and Ar⁴, can be used independently with the scope of any other instance of a variable substituent. As such, the invention includes combinations of the different aspects.

Unless specified otherwise, these terms have the following meanings. “Alkyl” means a straight or branched alkyl group composed of 1 to 6 carbons, preferably composed of 1 to 3 carbons. “Alkenyl” means a straight or branched alkyl group composed of 2 to 6 carbons with at least one double bond, preferably composed of 2 to 3 carbons. “Alkynyl” means a straight or branched alkyl group composed of 2 to 6 carbons with at least one triple bond, preferably composed of 2 to 4 carbons. “Cycloalkyl” means a monocyclic ring system composed of 3 to 7 carbons. “Haloalkyl” and “haloalkoxy” include all halogenated isomers from monohalo to perhalo. Terms with a hydrocarbon moiety (e.g. alkoxy) include straight and branched isomers for the hydrocarbon portion. Parenthetic and multiparenthetic terms are intended to clarify bonding relationships to those skilled in the art. For example, a term such as ((R)alkyl) means an alkyl substituent further substituted with the substituent R.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some of the compounds of the invention exist in stereoisomeric forms, one example which is shown below. The invention includes all stereoisomeric forms of the compounds including enantiomers and diastereromers. Methods of making and separating stereoisomers are known in the art.

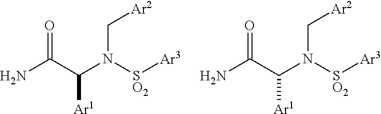

Some compounds of the invention are
α-[(4-Chlorobenzenesulfonyl)((4-t-butyloxycarbonylphenyl)methyl)amino]-3,5-difluorobenzeneacetamide;
α-[(4-Chlorobenzenesulfonyl)((4-carboxyphenyl)methyl)amino]-3,5-difluorobenzene-acetamide;
α-[(4-Chlorobenzenesulfonyl)((4-ethylaminocarbonylphenyl)methyl)amino]-3,5-difluorobenzeneacetamide;
α-[(4-Chlorobenzenesulfonyl)(4t-butylaminocarbonylphenylmethyl)amino]-3,5-difluorobenzeneacetamide;
α-[(4-Chlorobenzenesulfonyl)(4-azetidinylcarbonylphenylmethyl)amino]-3,5-difluorobenzene-acetamide;
α-[(4-Chlorobenzenesulfonyl)(4-methylaminocarbonylphenylmethyl)amino]-3,5-difluorobenzene-acetamide;
α-[(4-Chlorobenzenesulfonyl)((4-dimethylaminocarbonylphenylmethyl)amino]-3,5-difluorobenzene-acetamide;
α-[(4-Chlorobenzenesulfonyl)((4-cyclobutylaminocarbonylphenylmethyl)amino]-3,5-difluorobenzeneacetamide;
α-[(4-Chlorobenzenesulfonyl)(1-oxo-2-ethyl-1,2,3,4-tetrahydroisoquinolin-6-ylmethyl)amino]-3,5-difluorobenzeneacetamide;
α-[(4-Chlorobenzenesulfonyl)(4-imidazolylphenylmethyl)amino]-3,5-difluorobenzene-acetamide;
α-[(4-Chlorobenzenesulfonyl)(4-(1,2,4-triazolyl)phenylmethyl)amino]-3,5-difluorobenzene-acetamide;
α-[(4-Chlorobenzenesulfonyl)(4-pyrazolylphenylmethyl)amino]-3,5-difluorobenzene-acetamide;
α-[(4-Chlorobenzenesulfonyl)(4-pyridylmethyl)amino]-3,5-difluorobenzene-acetamide;
α-[(4-Chlorobenzenesulfonyl)(4-fluorophenylmethyl)amino]-3,5-difluorobenzene-acetamide;
α-[(4-Chlorobenzenesulfonyl)(4-trifluoromethylphenylmethyl)amino]-3,5-difluorobenzene-acetamide;
α-[(4-Chlorobenzenesulfonyl)(4-cyanophenylmethyl)amino]-3,5-difluorobenzene-acetamide;
α-[(4-chlorophenylsulfonyl)(4-(oxazol-2-yl)phenylmethyl)amino]-3,5-difluorobenzeneacetamide;
α-[(4-chlorophenylsulfonyl)(4-(4-(5-methyl-1,2,4-oxadiazol-3-yl)phenylmethyl)amino]-3,5-difluorobenzeneacetamide;
α-[(4-chlorophenylsulfonyl)(4-(4-(1,2,4-oxadiazol-3-yl)phenylmethyl)amino]-3,5-difluorobenzeneacetamide;
α-[(4-Chlorobenzenesulfonyl)((4-t-butyloxycarbonylphenyl)methyl)amino]-2,4-difluorobenzeneacetamide;
α-[(4-Chlorobenzenesulfonyl)((4-t-butyloxycarbonylphenyl)methyl)amino]-4-methoxybenzeneacetamide;
α-[(4-Chlorobenzenesulfonyl)((4-carboxyphenyl)methyl)amino]-2,4-difluorobenzeneacetamide;
α-[(4-Chlorobenzenesulfonyl)((4-carboxyphenyl)methyl)amino]-4-methoxybenzeneacetamide;
α-[(4-Chlorobenzenesulfonyl)((4-ethylaminocarbonylphenyl)methyl)amino]-2,4-difluorobenzene-acetamide;
α-[(4-Chlorobenzenesulfonyl)(4-t-butylaminocarbonylphenylmethyl)amino]-2,4-difluorobenzene-acetamide;
α-[(4-Chlorobenzenesulfonyl)((4-ethylaminocarbonylphenyl)methyl)amino]-4-methoxybenzene-acetamide;
α-[(4-Chlorobenzenesulfonyl)((4-ethylaminocarbonylphenyl)methyl)amino]-2-trifluoromethylbenzene-acetamide;
α-[(4-Chlorobenzenesulfonyl)(4-cyanophenylmethyl)amino]-2-trifluoromethyl-benzeneacetamide;
(R)-α-[(4-Chlorobenzenesulfonyl)((4-ethylaminocarbonylphenyl)methyl)amino]-2,4-benzene-acetamide;
(R)-α-[(4-Chlorobenzenesulfonyl)(1-oxo-2-ethyl-1,2,3,4-tetrahydroisoquinolin-6-ylmethyl)amino]-benzeneacetamide; and
α-[(4-Chlorobenzenesulfonyl)((4-ethylaminocarbonylphenyl)methyl)amino]-2-methylbenzene-acetamide.

### Synthetic Methods

Compounds of Formula I can be made according to methods known in the art including those described and illustrated in the schemes below. The formulas and variables illustrated in the synthetic methods section are intended only to assist describing the synthesis of Formula I compounds and are not to be confused with the variables used to define Formula I compounds in the claims or in other sections of the specification.

Abbreviations used in the schemes generally follow conventions used in the art. Chemical abbreviations used in the specification and examples are defined as follows: “NaHMDS” for sodium bis(trimethylsilyl)amide; “DMF” for N,N-dimethylformamide; “MeOH” for methanol; “NBS” for N-bromosuccinimide; “Ar” for aryl; “TFA” for trifluoroacetic acid; “LAH” for lithium aluminum hydride; “BOC”, “DMSO” for dimethylsulfoxide; “h” for hours; “rt” for room temperature or retention time (context will dictate); “min” for minutes; “EtOAc” for ethyl acetate; “THF” for tetrahydrofuran; “EDTA” for ethylenediaminetetraacetic acid; “Et₂O” for diethyl ether; “DMAP” for 4-dimethylaminopyridine; “DCE” for 1,2-dichloroethane; “ACN” for acetonitrile; “DME” for 1,2-dimethoxyethane; “HOBt” for 1-hydroxybenzotriazole hydrate; “DIEA” for diisopropylethylamine, “Nf” for CF₃(CF₂)₃SO₂—; and “TMOF” for trimethylorthoformate.

Some compounds of formula I can be prepared by the methods illustrated in Scheme 1. Compounds of formula 2 can be reacted with sulfonylating agents of formula Ar³SO₂Cl to generate compounds of formula 3. Compounds of formula 3 can be reacted with alkylating agents of formula X(CH₂)ₘAr² (where X═Br, Cl, I, O₃SCH₃, O₃S—C₆H₄—CH₃, O₃S—CF₃) to generate compounds of formula 1. Compounds of formula 3 can also be reacted with alcohols of formula HO(CH₂)ₘ Ar² in the presence of a dialkyl azodicarboxylate and a triaryl phosphine to provide compounds of formula 1. Compounds of formula 2 can also be reductively alkylated with aldehydes of formula OHC(CH₂)ₘ₋₁Ar² to provide compounds of formula 4. Compounds of formula 4 can be sulfonylated to generate compounds of formula 1.

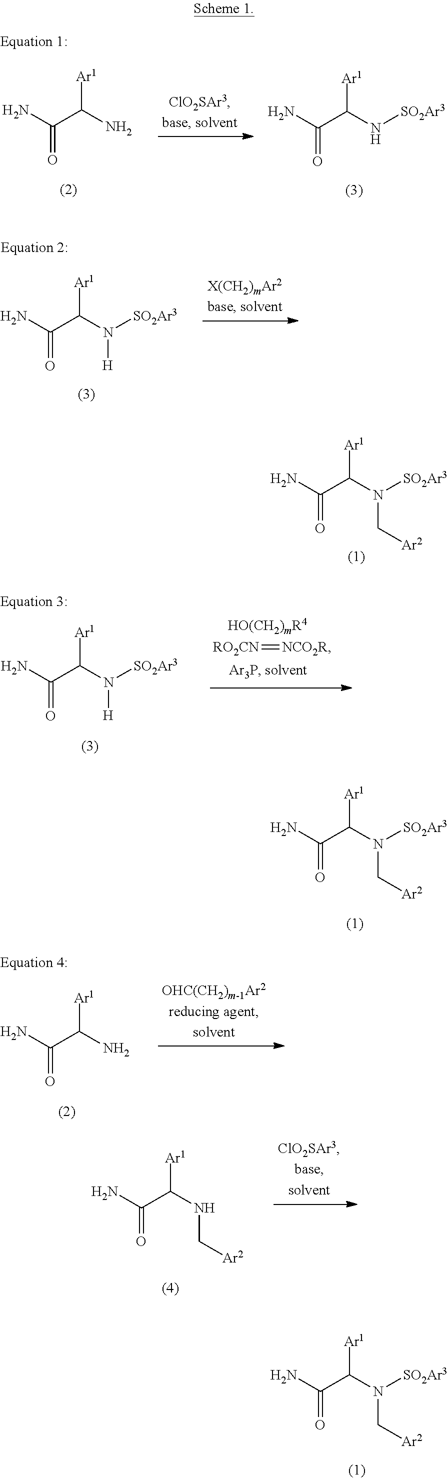

Some compounds of formula I can be prepared by the methods illustrated in Scheme 2. Compounds of formula 6 can be sulfonylated to generate compounds of formula 7. Compounds of formula 7 can be alkylated with agents of formula X(CH₂)ₘAr² (where X═Br, Cl, I, O₃SCH₃, O₃S—C₆H₄—CH₃, O₃S—CF₃) to generate compounds of formula 9. Compounds of formula 7 can also be reacted with alcohols of formula HO(CH₂)ₘ Ar² in the presence of a dialkyl azodicarboxylate and a triaryl phosphine to provide compounds of formula 9. Compounds of formula 6 can be reductively alkylated with aldehydes of formula OHC(CH₂)ₘ₋₁Ar² to provide compounds of formula 8. Compounds of formula 8 can be sulfonylated with agents of formula Ar³SO₂Cl to generate compounds of formula 9. Esters of formula 9 can be hydrolyzed to carboxylic acids of formula 10. Acids of formula 9 can be converted to amides of formula 1 by treatment with NH₄Cl or NH₃ in the presence of a coupling reagent and a base in an inert solvent. Some coupling reagents include 1-hydroxybenzotriazole (HOBt), 1-ethyl-3-(3-dimethylaminopropyl)carbodiimide (EDC), O-(7-azabenzotriazolyl)-N,N,N′,N′-tetramethyluronium hexafluorophosphate (HATU), benzotriazo-1-yloxytripyrrolidinophosphonium hexafluorophosphate (PyBOP), benzotriazo-1-yloxy-tris(dimethylamino)phosphonium hexafluorophosphate (BOP), and O-benzotraizol-1-yl-N,N,N′,N′-tetramethyluronium tetrafluoroborate (TBTU).

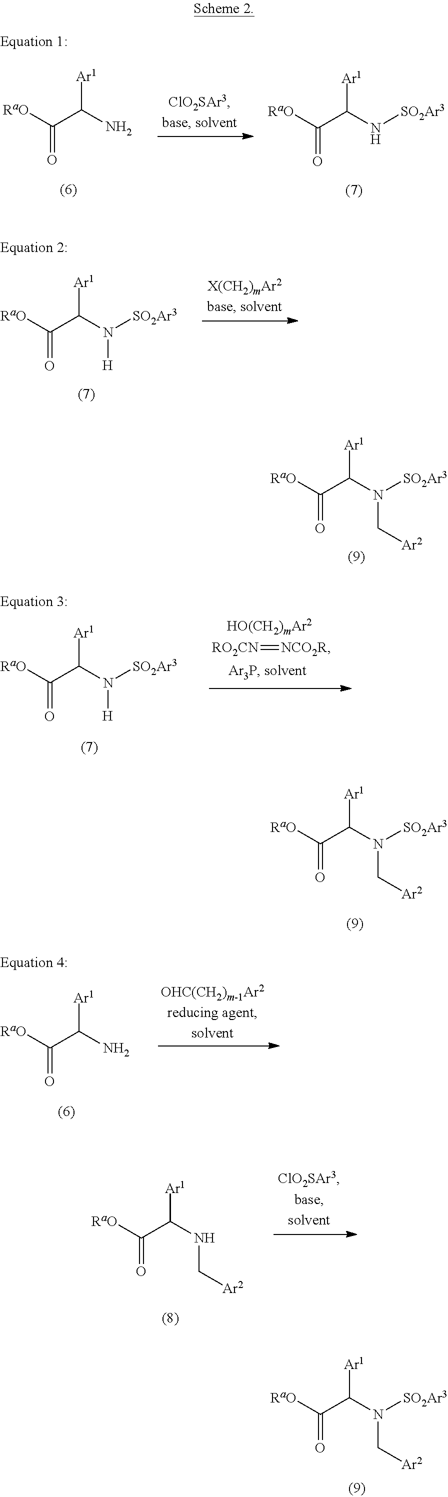

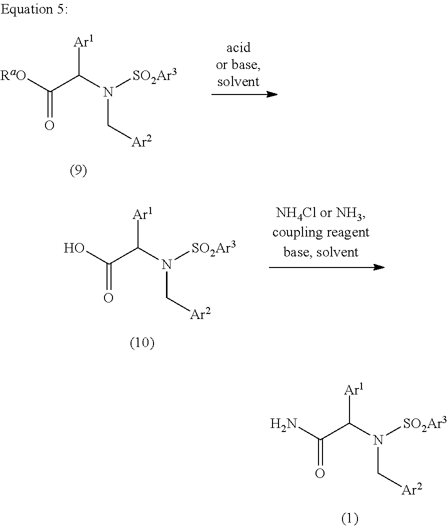

Some compounds of formula 2 or 3 can be prepared by the methods illustrated in Scheme 3. Esters of formula 11 can be brominated to give bromoesters of formula 12. Bromoesters of formula 12 can be converted to azides of formula 13. Azides of formula 13 can be transformed into protected amines of formula 14. Esters of formula 14 can be hydrolyzed to acids of formula 15. Compounds of formula 15 may be converted to primary amides of formula 16 by treatment with NH₄Cl or NH₃ in the presence of a coupling reagent. Compounds of formula 16 may be de-protected to afford compounds of formula 17. Alternatively, intermediates of formula 15 can be hydrolyzed to compounds of formula 17. Compounds of formula 17 may be sulfonylated to compounds of formula 18. Amides of formula 3 may be prepared from acids of formula 18 by treatment with NH₄Cl or NH₃ in the presence of a coupling reagent.

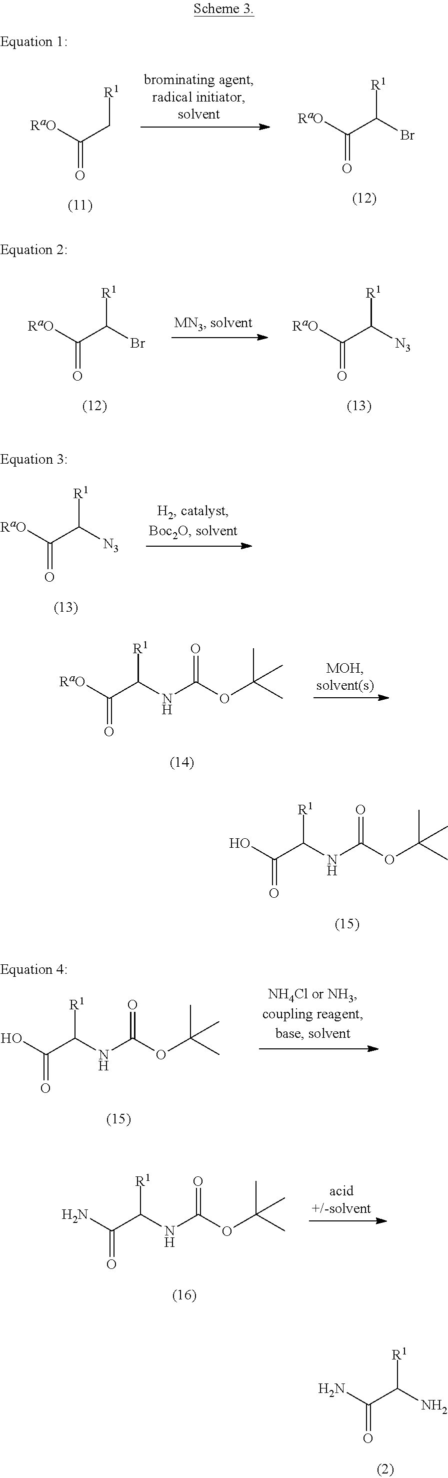

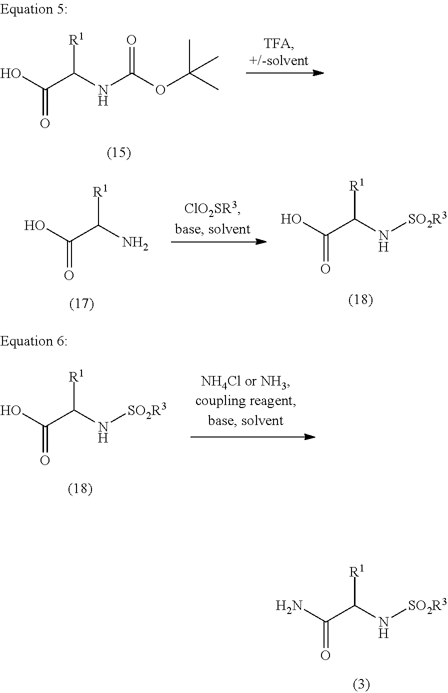

Some compounds of formula 2 can be prepared by the methods illustrated in Scheme 4. Boronic acids R¹B(OH)₂, glyoxylic acid hydrate and amines R^{b}R^{c}CHNH₂ can be reacted to provide intermediates of formula 19. Amides of formula 20 can be prepared from acids of formula 19 by treatment with NH₄Cl or NH₃ in the presence of a coupling reagent. Compounds of formula 2 can be prepared from amides of formula 20.

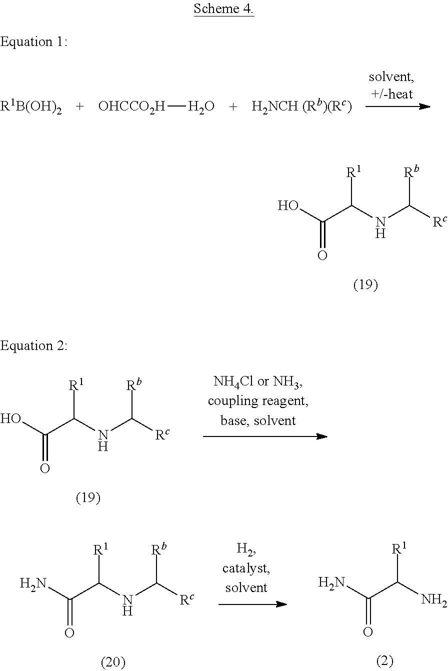

### Biological Methods

Competitive in vitro binding assays can be used to identify compounds that inhibit γ-secretase activity. For example, [³H]-Compound A can be used for binding assays with membranes from THP-1 cells (Seiffert, D. et al., J. Biol. Chem. 2000, 275, 34086). Compound A is described in U.S. patent U.S. Pat. No. 6,331,408; PCT Publication WO 00/28331; PCT Publication WO 00/07995; and J. Biol. Chem. 2000, 275, 34086.

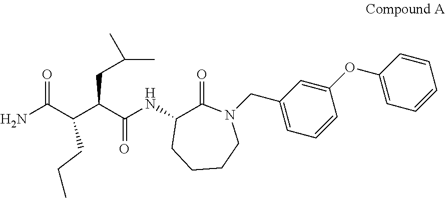

To evaluate compounds using this assay, THP-1 cells were grown in spinner cultures in RPMI 1640 containing L-glutamine and 10 μM β-mercaptoethanol to a density of 5×10⁵ cells/ml. Cells were harvested by centrifugation and cell pellets were quick frozen in dry ice/ethanol and stored at −70° C. prior to use. The pellets of approximately 2×10⁴ THP-1 cells were homogenized using a Brinkman Polytron at setting 6 for 10 sec. The homogenate was centrifuged at 48,000×g for 12 min, and the resulting pellet was washed by repeating the homogenization and centrifugation. The final cell pellet was resuspended in buffer to yield a protein concentration of approximately 0.5 mg/ml. Assays were initiated by the addition of 150 μl of membrane suspension to 150 μl of assay buffer containing 0.064 μCi of radioligand and various concentrations of unlabeled compounds. Binding assays were performed in duplicate in polypropylene 96-well plates in a final volume of 0.3 ml containing 50 mM Hepes, pH 7.0, and 5% dimethyl sulfoxide. Nonspecific binding was defined using incubations with 300 nM compound A (Seiffert, D. et al., J. Biol. Chem. 2000, 275, 34086). After incubating at 23° C. for 1.3 hr, bound ligand was separated from free radioligand by filtration over GFF glass fiber filters presoaked in 0.3% ethyleneimine polymer solution. Filters were washed three times with 0.3 ml of ice cold phosphate-buffered saline, pH 7.0, containing 0.1% Triton X-100. Filter-bound radioactivity was measured by scintillation counting. IC₅₀ values were then determined and used to calculate K₁ values using the Cheng-Prusoft correction for IC₅₀ values. Compounds were scored as active γ-secretase inhibitors if K₁ values were less than 10 μM.

γ-Secretase inhibitors were also evaluated using in vitro assays based on the inhibition of Aβ formation in cultured cells. Cultured human cell lines, such as HEK293 and H4 cells, which express APP and γ-secretase activity or transfected derivative cell lines that overexpress wild-type APP, mutant APP, or APP fusion proteins will secrete Aβ peptides into the culture media that can be quantified as previously outlined (Dovey, H. et al., J. Neurochem. 2001, 76, 173). The incubation of these cultured cells with γ-secretase inhibitors decreases the production of Aβ peptides. For instance, H4 cells stably transfected to overexpress the HPLAP-APP fusion protein described above were grown as above, detached, and adjusted to 2×105 cells/ml. 100 μl of the resulting suspension was then added to each well of a 96-well plate. After 4 hrs, the media was removed and replaced with 100 μl serum-free media containing various dilutions of the test compound. Plates were then incubated for 18 hrs at 37° C. and a 100 μl aliquot of the tissue culture supernatant was removed for determination of Aβ levels using time-resolved fluorescence of the homogenous sample as outlined above. The extent of Aβ inhibition was used to calculate the IC₅₀ value for the test compound. Compounds are considered active when tested in the above assay if the IC₅₀ value for the test compound is less than 50 μM.

Representative compounds were evaluated in the above assay and were determined to inhibit Aβ formation. Results are summarized in Table 1.

TABLE 1
Inhibition of β-amyloid
peptide formation in human H4 cells.

| | Binding |
|---|---|
| | affinity |
| Example | (IC₅₀ in nM) |
| 1 | +++ |
| 2 | ++ |
| | (116) |
| 3 | +++ |
| | (1.28) |
| 4 | +++ |
| 5 | +++ |
| 6 | +++ |
| 8 | +++ |
| | (0.93) |
| 9 | +++ |
| 11 | +++ |
| 12 | +++ |
| | (9.15) |
| 14 | +++ |
| 16 | +++ |
| | (12.4) |
| 18 | +++ |
| 19 | +++ |
| 31 | ++ |
| | (127) |

Activity (IC₅₀): 0.25-50 nM = +++; 50-500 nM = ++; 500-10000 nM = +.

In addition to cleaving APP, γ-secretase cleaves other substrates. These include the Notch family of transmembrane receptors (see Selkoe, D. Physiol. Rev. 2001, 81, 741; Wolfe, M. J. Med. Chem. 2001, 44, 2039); LDL receptor-related protein (May, P. et al. J. Biol. Chem. 2002, 277, 18736); ErbB-4 (Ni, C. Y. et al. Science 2001, 294, 2179); E-cadherin (Marambaud, P. et al., EMBO J. 2002, 21, 1948); and CD44 (Okamoto, I. et al., J. Cell Biol. 2001, 155, 755). If inhibition of cleavage of non-APP substrates causes undesirable effects in humans, then desired γ-secretase inhibitors would preferentially inhibit APP cleavage relative to unwanted substrates. Notch cleavage can be monitored directly by measuring the amount of cleavage product or indirectly by measuring the effect of the cleavage product on transcription (Mizutani, T. et al. Proc. Natl. Acad. Sci. USA 2001, 98, 9026).

### Pharmaceutical Composition and Methods of Use

“Therapeutically effective” means the amount of agent required to provide a meaningful patient benefit as understood by practitioners in the field of amyloids or Alzheimer's disease.

“Patient” means a person suitable for therapy as understood by practitioners in the field of amyloids or Alzheimer's disease.

“Treatment,” “therapy,” “regimen,” “HCV infection,” and related terms are used as understood by practitioners in the field of amyloids or Alzheimer's disease.

Another aspect of this invention includes pharmaceutical compositions comprising at least one compound of formula I in combination with at least one pharmaceutical adjuvant, carrier, or diluent.

Another aspect of this invention relates to a method of treatment of disorders characterized by aberrant extracellular deposition of amyloid and which are responsive to the inhibition of β-amyloid peptide in a patient in need thereof, which comprises administering a therapeutically effective amount of a compound of formula I or a nontoxic pharmaceutically acceptable salt thereof.

Another aspect of this invention relates to a method for treating systemic (vascular) amyloidosis, pulmonary or muscle amyloidosis, Alzheimer's Disease, Down's Syndrome, or other diseases characterized by extracellular amyloid deposition in a patient in need thereof, which comprises administering a therapeutically effective amount of a compound of Formula I or a pharmaceutically acceptable salt thereof.

The compounds are generally given as pharmaceutical compositions comprised of a therapeutically effective amount of a compound of Formula I, or a pharmaceutically acceptable salt, and a pharmaceutically acceptable carrier and may contain conventional excipients. A therapeutically effective amount is the amount needed to provide a meaningful patient benefit as determined by practitioners in that art. Pharmaceutically acceptable carriers are those conventionally known carriers having acceptable safety profiles. Compositions encompass all common solid and liquid forms including capsules, tablets, losenges, and powders as well as liquid suspensions, syrups, elixers, and solutions. Compositions are made using common formulation techniques and conventional excipients (such as binding and wetting agents) and vehicles (such as water and alcohols). See, for example, Remington's Pharmaceutical Sciences, Mack Publishing Company, Easton, Pa., 17th edition, 1985.

Solid compositions are normally formulated in dosage units providing from about 1 to about 1000 mg of the active ingredient per dose. Some examples of solid dosage units are 1 mg, 10, mg, 100, mg, 250 mg, 500 mg, and 1000 mg. Liquid compositions are generally in a unit dosage range of 1-100 mg/mL. Some examples of liquid dosage units are 1 mg/mL, 10 mg/mL, 25, mg/mL, 50 mg/mL, and 100 mg/mL.

The invention encompasses all conventional modes of administration; oral and parenteral methods are preferred. Typically, the daily dose will be 0.01-100 mg/kg body weight daily. Generally, more compound is required orally and less parenterally. The specific dosing regime, however, should be determined by a physician using sound medical judgement.

### DESCRIPTION OF SPECIFIC EMBODIMENTS

Abbreviations used in the schemes generally follow conventions used in the art. Chemical abbreviations used in the specification and Examples are defined as follows: “NaHMDS” for sodium bis(trimethylsilyl)amide; “DMF” for N,N-dimethylformamide; “MeOH” for methanol; “NBS” for N-bromosuccinimide; “Ar” for aryl; “TFA” for trifluoroacetic acid; “LAH” for lithium aluminum hydride; “BOC”, “DMSO” for dimethylsulfoxide; “h” for hours; “rt” for room temperature or retention time (context will dictate); “min” for minutes; “EtOAc” for ethyl acetate; “THF” for tetrahydrofuran; “EDTA” for ethylenediaminetetraacetic acid; “Et₂O” for diethyl ether; “DMAP” for 4-dimethylaminopyridine; “DCE” for 1,2-dichloroethane; “ACN” for acetonitrile; “DME” for 1,2-dimethoxyethane; “HOBt” for 1-hydroxybenzotriazole hydrate; “DIEA” for diisopropylethylamine, “Nf” for CF₃(CF₂)₃SO₂—; and “TMOF” for trimethylorthoformate.

Abbreviations generally follow convention: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “min” for minute or minutes, “h” for hour or hours, “rt” for room temperature, “RT” for retention time, “atm” for atmosphere, “psi” for pounds per square inch, “conc.” for concentrate, “sat” or “sat'd” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

Analytical data were generated using the following procedures. Proton NMR spectra were recorded on an Varian FT-NMR (300 MHz or 500 MHz); chemical shifts were recorded in ppm (δ) from an internal tetramethysilane standard in deuterochloroform or deuterodimethylsulfoxide as specified below. Mass spectra (MS) or high resolution mass spectra (HRMS) were recorded on a Finnegan MAT 8230 spectrometer (using electrospray ionization (ES, + or −) or atmospheric chemi-ionization (APCI, + or −) with NH₃ as the carrier gas). Melting points were recorded on a Buchi Model 510 melting point apparatus and are uncorrected. Boiling points are uncorrected. All pH determinations during workup were made with indicator paper. Combustion analyses were performed by Quantitative Technologies, Whitehouse, N.J.

Reagents were purchased from commercial sources and, where necessary, purified prior to use. Chromatography (thin layer (TLC), flash or preparative) was performed on silica gel 60 using the solvent systems indicated below. Analytical purity was routinely assessed on a Shimadzu Model 8A HPLC using reverse phase conditions (MeOH:H₂O:TFA::10:90:0.1 to 90:10:0.1)(flow rate=4 mL/min, wavelength=220 nm, gradient time=3 min. Preparative reverse phase high pressure liquid chromatography (HPLC) was performed on a Varian-Rainin model SD-200 machine using the solvent conditions enumerated below in the individual examples. Chiral chromatography was performed on a Shimadzu model LC-8A HPLC as described below for the individual examples. For mixed solvent systems, the volume ratios are given. Otherwise, parts and percentages are by weight.

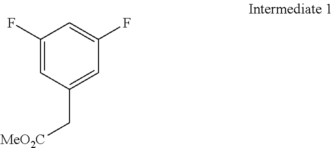

Methyl 3,5-Difluorobenzeneacetate. 3,5-Difluorophenylacetic acid (75 g, 0.44 mol) was dissolved in methanol (600 mL) and the resulting solution was cooled to 0° C. with stirring. Thionyl chloride (95 mL, 1.31 mol) was added dropwise over 30 min. The reaction mixture was then warmed to reflux temperature and stirred for 3 h. The reaction mixture was then concentrated in vacuo. The residue was taken up in toluene and concentrated in vacuo again. This residue was taken up in ether and the resulting solution was washed three times with a saturated NaHCO₃ solution, dried over MgSO₄ and filtered. Solvent was removed in vacuo to afford the title product (80.9 g, 99% yield): ¹H NMR (CDCl₃, 300 MHz): 6.81 (dt, 2H, J=8, 1), 6.72 (td, 1H, J=8, 1), 3.71 (s, 3H), 3.60 (s, 2H); HRMS (ES): Calcd for C₉H₇F₂O₂ (M⁺−H): 185.0414, Found: 185.0420.

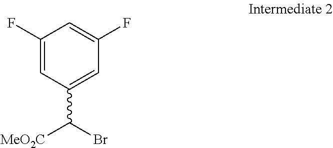

Methyl α-Bromo-3,5-Difluorobenzeneacetate. Methyl 3,5-difluorophenylacetate (35 g, 188 mmol), N-bromosuccinimide (36.1 g, 207 mmol), AIBN (3.1 g, 18.8 mmol) and dry CCl₄ (700 mL). The mixture was heated to reflux temperature and stirred under a nitrogen atmosphere for 18 h. The reaction mixture was then cooled to ambient temperature and filtered through Celite. The filtrate was concentrated in vacuo to give a yellow oil. Column chromatography (CH₂Cl₂) afforded three fractions after removal of solvent in vacuo: (1) the title product (23 g, 46% yield, R_{f}=0.75): ¹H NMR (CDCl₃, 300 MHz): 7.06 (dt, 2H, J=8, 2), 6.78 (td, 1H, J=8, 2), 5.23 (s, 1H), 3.78 (s, 3H); MS (ES): 263, 265 (C₉H₆BrF₂O₂, M⁺−H); (2) a mixture of the title product and starting ester, a yellow oil (14.7 g, R_{f}=0.75 and 0.6) and (3) starting ester (1.1 g, R_{f}=0.6).

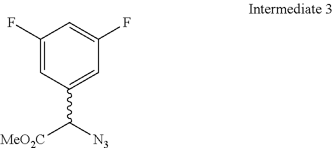

Methyl α-Azido-3,5-Difluorobenzeneacetate. Methyl bromo-(3,5-difluorophenyl)acetate (23 g, 87 mmol), sodium azide (11.3 g, 174 mmol) and dry CH₃CN (240 mL) were mixed and stirred at room temperature under a nitrogen atmosphere for 20.5 h. The reaction mixture was concentrated to a yellow slurry, which was taken up in EtOAc (200 mL). Three washings with water, one with brine, drying over MgSO₄ and filtration gave a yellow solution. Removal of solvent in vacuo provided a clear orange liquid, which was used without further purification: (19.2 g): ¹H NMR (CDCl₃, 300 MHz): 6.92 (dt, 2H, J=8, 1), 6.80 (td, 1H, J=8, 1), 4.96 (s, 1H), 3.77 (s, 3H); IR (film, NaCl, cm⁻¹): 3092 (w), 2995 (w), 2959 (w), 2848 (w), 2114 (s), 1750 (s), 1700 (m), 1625 (s), 1601 (s), 1506 (w), 1464 (m), 1438 (m), 1325 (s), 1265 (m), 1218 (s), 1208 (m), 1177 (m), 1123 (s), 992 (m).

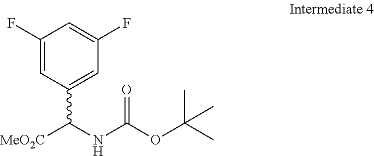

α-[[(1,1-Dimethylethoxy)carbonyl]amino]-3,5-difluorobenzeneacetic acid, methyl ester. Nitrogen gas was bubbled through a solution of di-tert-butylcarbonate (11.6 g, 53 mmol) in EtOAc (55 mL) in a Parr apparatus bottle. Palladium catalyst (10% on carbon, 4.3 g) was added carefully. The reaction bottle was charged with nitrogen gas after three repetitive evacuations, then it was charged with hydrogen gas after one evacuation. The bottle was shaken under a pressure ≦50 psi for 1 h. The bottle was evacuated again and hydrogen gas was replaced with nitrogen. A solution of methyl azido-(3,5-difluorophenyl)acetate (10 g, 44 mmol) in EtOAc (55 mL, saturated with N₂ as before) was added. Hydrogenation was resumed at a pressure ≦50 psi for 18 h. Hydrogen was replaced with nitrogen. The black suspension was filtered through Celite. The filtrate was washed twice with a saturated NaHSO₄ solution, twice with a saturated NaHCO₃ solution and once with brine. The organic solution was dried over MgSO₄ and filtered. Solvent was removed in vacuo to provide the title product (12.7 g, 96% yield), which was used without further purification: ¹H NMR (CDCl₃, 300 MHz): 6.92 (d, 2H, J=8), 6.75 (t, 1H, J=8), 5.70 (s, 1H), 5.35 (m, 1H), 3.74 (s, 3H), 1.52 (s, 6H), 1.43 (s, 3H); HRMS (ES): Calcd for C₁₄H₁₆F₂NO₄ (M⁺−H): 300.1047, Found: 300.1053.

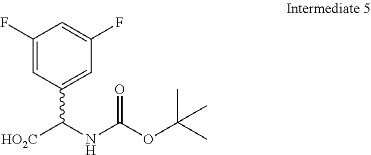

α-[[(1,1-Dimethylethoxy)carbonyl]amino]-3,5-difluorobenzeneacetic acid. α-[[(1,1-Dimethylethoxy)carbonyl]amino]-3,5-difluorobenzeneacetic acid, methyl ester (12.7 g, 42.3 mmol) was dissolved in a mixture of THF (150 mL) and MeOH (25 mL). The resulting solution was cooled to 0° C. with stirring. A solution of LiOH (1.52 g, 63.4 mmol) in water (50 mL) was added dropwise with stirring and the reaction mixture was warmed to ambient temperature over 3 h. Solvent was removed in vacuo and the residue was taken up in EtOAc (200 mL). The organic mixture was washed with a 5% NaHSO₄ solution (40 mL) twice and brine (40 mL) twice. The organic solution was dried over MgSO₄ and filtered. Solvent was removed in vacuo to give the title product (8.03 g, 66% yield): ¹H NMR (MeOH-d₄, 300 MHz): 7.04 (m, 2H), 6.90 (m, 1H), 5.23 (s, 1H), 1.43 (m, 9H); HRMS (ES): Calcd for C₁₃H₁₄F₂NO₄ (M⁺−H): 286.0891, Found: 286.0901.

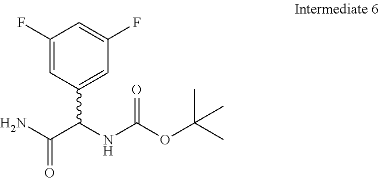

α-[[(1,1-Dimethylethoxy)carbonyl]amino]-3,5-difluorobenzeneacetamide. α-[[(1,1-Dimethylethoxy)carbonyl]amino]-3,5-difluorobenzeneacetic acid (8.0 g, 27.9 mmol) was dissolved in DMF (180 mL) and the solution was cooled with stirring to 0° C. under a nitrogen atmosphere. N,N′-Diisopropyl-N-ethylamine (7.3 mL, 41.8 mmol) was added, followed by O-(7-azabenzotriazol-1-yl)-N,N,N′,N′-tetramethyluronium hexafluorophosphate (HATU, 15.9 g, 41.8 mmol). Stirring at 0° C. was continued for 30 min Ammonia gas was bubbled through the reaction mixture until a suspension formed (˜5 min). The reaction mixture was warmed to ambient temperature with stirring over 18 h. Dilution with EtOAc (500 mL) gave a solution, which was washed with water (25 mL) three times, a 5% NaHSO₄ solution (30 mL) three times, a saturated NaHCO₃ solution twice, a 5% LiCl solution (50 mL) three times) and brine once. The organic solution was dried over MgSO₄ and filtered. Solvent was removed in vacuo to provide the title product (7.13 g, 89% yield) which was used without further purification: ¹H NMR (MeOH-d₄, 300 MHz): 7.04 (m, 2H), 6.90 (m, 1H), 5.17 (s, 1H), 1.43 (m, 9H); HRMS (ES): Calcd for C₁₃H₁₂F₂N₂O₃ (M⁺+H): 287.1207, Found: 287.1212.

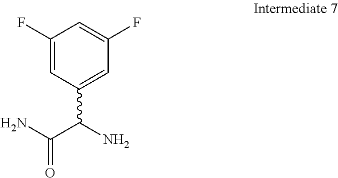

α-Amino-3,5-difluorobenzeneacetamide. α-[[(1,1-Dimethylethoxy)carbonyl]amino]-3,5-difluorobenzeneacetamide (7.1 g, 24.8 mmol), TFA (20.1 mL, 260.4 mmol) and CH₂Cl₂ (150 mL) were stirred at ambient temperature under a nitrogen atmosphere for 5 h. Solvent was removed in vacuo to afford. The residue was dissolved in EtOAc (150 mL) and the resulting solution was washed with a saturated K₂CO₃ solution (40 mL) three times, and brine once. Drying over Mg₂SO₄, filtration and concentration of the filtrate in vacuo provided the title product (a solid, 4.36 g, 94% yield) which was used without further purification: ¹H NMR (MeOH-d₄, 300 MHz): 7.04 (m, 2H), 6.86 (m, 1H), 4.66 (s, 1H), 1.43 (m, 3H, concentration dependent); HRMS (ES): Calcd for C₈H₉F₂N₂O (M⁺+H): 187.0683, Found: 187.0698.

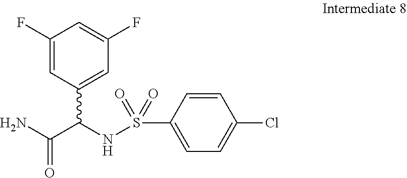

α-[4-Chlorobenzenesulfonylamino]-3,5-difluorobenzeneacetamide. α-Amino-3,5-difluorobenzeneacetamide (4.33 g, 23.3 mmol) was dissolved in CH₃CN (125 mL) and the solution was cooled to 0° C. with stirring. Triethylamine (11.4 mL, 81.4 mmol) was added, followed by 4-chlorobenzenesulfonyl chloride (4.91 g, 23.0 mmol). The reaction mixture was warmed to ambient temperature over 50 h. Solvent was removed in vacuo. The residue was dissolved in EtOAc (200 mL). The solution was washed successively with a 5% NaHSO₄ solution (30 mL) twice, a saturated NaHCO₃ solution (30 mL) twice and brine (25 mL) twice. The organic layer was dried over MgSO₄ and filtered. Solvent was removed in vacuo to afford the title product (white solid, 71.0 g, 85% yield) which was used without further purification: ¹H NMR (MeOH-d₄, 300 MHz): 7.74 (d, 2H, J=9), 7.46 (d, 2H, J=9), 6.86 (m, 3H), 5.00 (s, 1H), 1.40 (m, 3H, concentration dependent); HRMS (ES⁺): Calcd for C₁₄H₁₂ClF₂N₂O₃S (M⁺+H): 361.0237, Found: 361.0225.

Separation of enantiomers of α-(4-Chlorobenzenesulfonylamino)-3,5-difluorobenzeneacetamide. Chiral chromatography (1.0 g) (Chiralcel AD column (5×50 cm, 20 μm), heptane:iPrOH::7:3 at 70.0 mL/min, Shimadzu Model LC-8A high pressure preparative liquid chromatograph (HPLC) gave two enantiomers after removal of solvent in vacuo.

Intermediate 8a: Low retention time enantiomer (retention time=32 min, 250 mg): ¹H NMR (MeOH-d₄, 300 MHz): 7.72 (d, 2H, J=9), 7.44 (d, 2H, J=9), 6.82 (m, 3H), 4.97 (s, 1H), 4.83 (m, 3H); HRMS (ES⁻): Calcd for C₁₄H₁₀ClF₂N₂O₃S (M⁺−H): 359.0069, Found: 359.0006.

Intermediate 8b: High retention time enantiomer (retention time=48 min, 157 mg): ¹H NMR (MeOH-d₄, 300 MHz): 7.72 (d, 2H, J=9), 7.44 (d, 2H, J=9), 6.82 (m, 3H), 4.97 (s, 1H), 4.83 (m, 3H); HRMS (ES⁻): Calcd for C₁₄H₁₀ClF₂N₂O₃S (M⁺−H): 359.0069, Found: 359.0071.

Following the procedure described for Intermediate 5, intermediates 9-10 were synthesized from the appropriate amino acid.

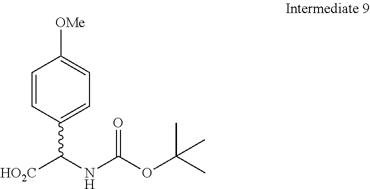

α-[[(1,1-Dimethylethoxy)carbonyl]amino]-4-methoxybenzeneacetic acid. 3.25 g (42% yield): ¹H-NMR (MeOH-d₄, 300 MHz): 7.31 (d, 2H, J=8), 6.90 (d, 2H, J=8), 5.10 (s, 1H), 4.80 (m, 2H, concentration dependent), 3.78 (s, 3H), 1.42 (s, 9H); HRMS (ES⁻): Calcd for C₁₄H₁₈NO₅ (M⁺−H): 280.1185, Found: 287.1176.

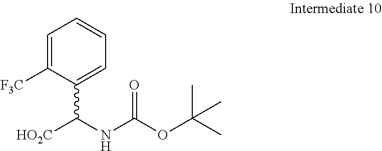

α-[[(1,1-Dimethylethoxy)carbonyl]amino]-2-trifluoromethylbenzeneacetic acid. 5.24 g (72% yield): ¹H-NMR (MeOH-d₄, 300 MHz): 7.73 (d, 1H, J=9), 7.56 (m, 3H), 5.60 (s, 1H), 4.80 (m, 2H, concentration dependent), 1.40 (s, 9H); HRMS (ES⁻): Calcd for C₁₄H₁₅F₃NO₄ (M⁺−H): 318.0953, Found: 318.0961.

Following the procedure outlined for Intermediate 6, intermediates 11-13 were prepared from the appropriate benzeneacetic acid derivative.

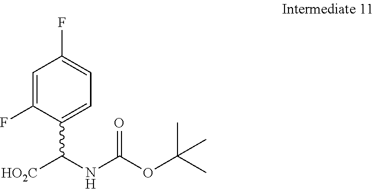

α-[[(1,1-Dimethylethoxy)carbonyl]amino]-2,4-difluorobenzeneacetic acid. α-Amino-2,4-difluorobenzeneacetic acid (5 g, 26.7 mmol) was dissolved with stirring in a mixture of water (50 mL) and dioxane (50 mL). The reaction mixture was cooled to 0° C. and Et₃N (18.6 mL, 133.6 mmol) was added, followed by di-t-butyldicarbonate (8.75 g, 40.0 mmol). The reaction mixture was stirred while warming to room temperature over 18 h. Solvent was removed in vacuo. The residue was taken up in EtOAc (100 mL). The organic solution was washed twice with a 5% NaHSO₄ solution (20 mL) and twice with brine (20 mL). The organic solution was dried over MgSO₄ and filtered. Removal of solvent in vacuo afforded the title product (5.31 g): ¹H-NMR (MeOH-d₄, 300 MHz): 7.42 (m, 1H), 6.97 (m, 2H), 5.46 (s, 1H), 4.80 (m, 2H, concentration dependent), 1.43 (s, 9H); HRMS (ES): Calcd for C₁₃H₁₄F₂NO₄ (M⁺−H): 286.0891, Found: 286.0900.

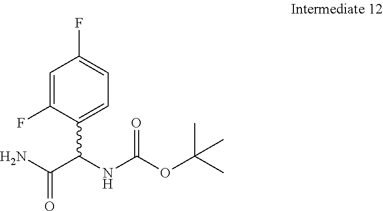

α-[[(1,1-Dimethylethoxy)carbonyl]amino]-2,4-difluorobenzeneacetamide. 4.07 g (78% yield): ¹H-NMR (MeOH-d₄, 300 MHz): 7.42 (m, 1H), 6.96 (m, 2H), 5.46 (s, 1H), 4.80 (m, 3H, concentration dependent), 1.41 (s, 9H); HRMS (ES⁺): Calcd for C₁₃H₁₇F₂N₂O₃ (M⁺+H): 286.1268, Found: 287.1221.

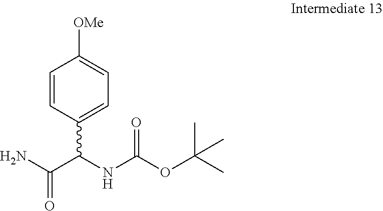

α-[[(1,1-Dimethylethoxy)carbonyl]amino]-4-methoxybenzeneacetamide. 2.48 g (78% yield): ¹H-NMR (MeOH-d₄, 300 MHz): 7.32 (d, 2H, J=8), 6.89 (d, 2H, J=8), 5.07 (s, 1H), 4.80 (m, 3H, concentration dependent), 3.78 (s, 3H), 1.42 (s, 9H); HRMS (ES⁺): Calcd for C₁₄H₂₁N₂O₄ (M⁺+H): 281.1501, Found: 281.1505.

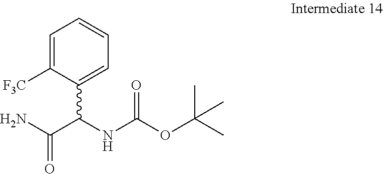

α-[[(1,1-Dimethylethoxy)carbonyl]amino]-2-trifluoromethylbenzeneacetamide. 4.87 g (98% yield): ¹H-NMR (MeOH-d₄, 300 MHz): 7.90 (s, 1H), 7.30 (s, 2H), 7.62 (m, 2H), 7.52 (s, 2H), 5.56 (s, 1H), 1.44 (s, 9H); HRMS (ES⁺): Calcd for C₁₄H₁₈F₃N₂O₃ (M⁺+H): 319.1268, Found: 319.1275.

Following the procedure outlined for intermediate 7, examples 15-17 were prepared from the appropriate benzeneacetamide derivative.

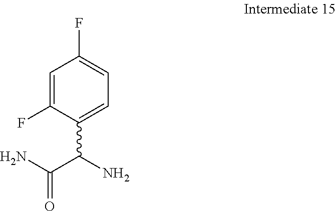

α-Amino-2,4-difluorobenzeneacetamide. 2.14 g (82% yield): ¹H-NMR (MeOH-d₄, 300 MHz): 7.59 (m, 1H), 6.94 (m, 2H), 4.80 (m, 4H, concentration dependent), 4.70 (s, 1H); HRMS (ES⁺): Calcd for C₈H₉F₂N₂O (M⁺+H): 187.0678, Found: 187.0684.

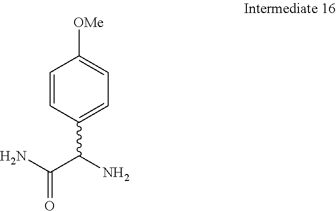

α-Amino-4-methoxybenzeneacetamide. 1.05 g (58% yield): ¹H-NMR (MeOH-d₄, 300 MHz): 7.33 (d, 2H, J=8), 6.90 (d, 2H, J=8), 4.80 (m, 4H, concentration dependent), 4.40 (s, 1H), 3.78 (s, 3H); HRMS (ES⁺): Calcd for C₉H₁₃N₂O₂ (M⁺+H): 181.0977, Found: 181.0983.

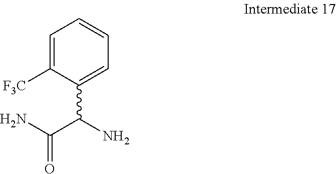

α-Amino-4-trifluoromethylbenzeneacetamide. 2.87 g (86% yield): ¹H-NMR

(MeOH-d₄, 300 MHz): 7.65 (m, 3H), 7.48 (t, 1H, J=8), 4.80 (m, 5H); HRMS (ES⁺): Calcd for C₉H₁₀F₃N₂O (M⁺+H): 219.0759, Found: 219.0741.

Following the procedure outlined for Intermediate 8, intermediates 18-21 were prepared from the appropriate benzeneacetamide and 4-chlorobenzenesulfonyl chloride.

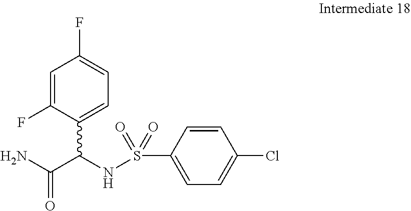

α-[4-Chlorobenzenesulfonylamino]-2,4-difluorobenzeneacetamide. 3.84 g (94% yield): ¹H-NMR (MeOH-d₄, 300 MHz): 7.72 (d, 2H, J=9), 7.44 (d, 2H, J=9), 7.27 (m, 1H), 6.81 (m, 2H), 5.17 (s, 1H), 4.85 (m, 3H), concentration dependent); HRMS (ES⁺): Calcd for C₁₄H₁₅ClF₂N₃O₃S (M⁺+NH₄): 378.0508, Found: 378.0480.

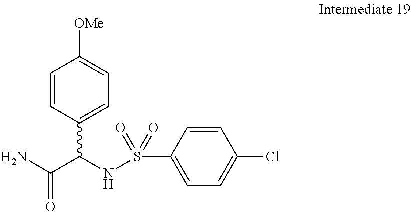

α-[4-Chlorobenzenesulfonylamino]-4-methoxybenzeneacetamide. 1.96 g (97% yield): ¹H-NMR (DMSO-d₆, 300 MHz): 8.49 (d, 1H, J=9), 7.65 (d, 2H, J=9), 7.50 (s, 1H), 7.48 (d, 2H, J=9), 7.17 (d, 2H, J=9), 7.05 (s, 1H), 6.74 (d, 2H, J=9), 4.86 (d, 1H, J=9), 3.69 (s, 3H); HRMS (ES⁺): Calcd for C₁₅H₁₉ClN₃O₄S (M⁺+NH₄): 372.0770, Found: 372.0794.

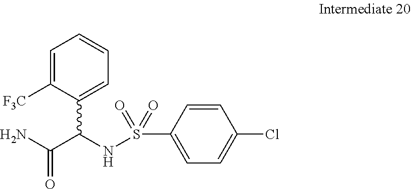

α-[4-Chlorobenzenesulfonylamino]-2-trifluoromethylbenzeneacetamide. 4.92 g (98% yield): ¹H-NMR (acetone-d₆, 300 MHz): 7.64 (d, 2H, J=9), 7.59 (d, 1H, J=9), 7.54 (d, 1H, J=9), 7.45 (m, 2H), 7.37 (d, 2H, J=9), 5.33 (s, 1H), 4.79 (m, 3H); HRMS (ES⁺): Calcd for C₁₅H₁₃ClF₃N₂O₃S (M⁺+H): 393.0269, Found: 393.0299.

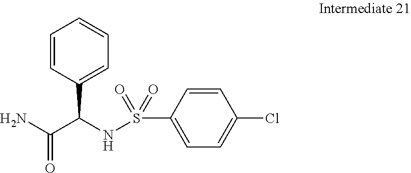

(R)-α-[4-Chlorobenzenesulfonylamino]-benzeneacetamide. 5.84 g (95% yield, 96% ee (Chiralcel OD, 4.6×50 mm, hexane:EtOH::10:90, 1 ml/min): ¹H-NMR (MeOH-d₄, 300 MHz): 7.72 (d, 2H, J=9), 7.44 (d, 2H, J=9), 7.26 (s, 5H), 6.81 (m, 2H), 4.93 (s, 1H), 4.85 (m, 3H), concentration dependent).

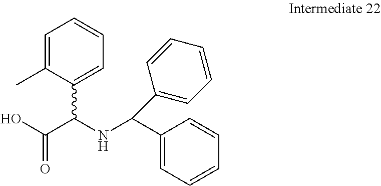

α-[Diphenylmethyl)amino]-2-methylbenzeneacetic acid. A mixture of 2-methylphenylboronic acid (4.08 g, 30 mmol), glyoxylic acid monohydrate (2.76 g, 30 mmol), aminodiphenylmethane (5.49 g, 30 mmol) in DCM (200 mL) was stirred at ambient temperature. Nitrogen gas was bubbled through the mixture for 15 min and the reaction flask was sealed with a septum cap. Stirring was continued for 150 h. Solvent was removed in vacuo. The residue was taken up in water (200 mL) and the mix was heated at reflux temperature for 30 with vigorous stirring. The mixture was cooled to room temperature and filtered. The collected solid was washed with copious amounts of water, then ether. The off-white solid was dried in vacuo (8.0 g, 80% yield): ¹H NMR (DMSO-d₆, 300 MHz): 7.9 (m, 2H), 7.25 (m, 14H), 4.71 (s, 1H), 1.98 (s, 3H); HRMS (ES⁺): 332 (M⁺+H).

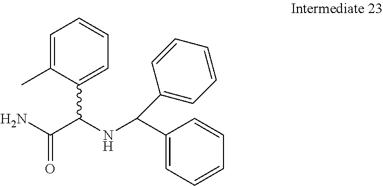

α-[Diphenylmethyl)amino]-2-methylbenzeneacetamide. A mixture of α-[diphenylmethyl)amino]-2-methylbenzeneacetic acid (8.0 g, 24.2 mmol), EDC (6.92 g, 36.1 mmol), HOBt (4.87 g, 36.1 mmol), iPr₂NEt (12.6 g, 17.0 mL<97.4 mmol) in DMF (107 mL) was stirred at ambient temperature under a nitrogen atmosphere. Ammonium chloride (2.71 g, 50.5 mmol) was added. Stirring was continued for 138 h. The reaction mixture was poured onto water (600 mL) and mixed. Three extractions with EtOAc (100 mL) were performed. The combined organic layers were washed with a 5% LiCl solution (50 mL) three times, then with brine (50 mL) twice. The organic solution was dried over MgSO₄ and filtered. Solvent was removed in vacuo to give a yellow oil. Column chromatography (EtOAc:hexane::1:1) and removal of solvent in vacuo provided the title product as a pale yellow glass (3.6 g, 45% yield): ¹H NMR (DMSO-d₆, 300 MHz): 7.24 (m, 14H), 6.70 (s, 1H), 5.4 (s, 1H), 4.78 (s, 1H), 4.40 (s, 1H), 2.10 (s, 3H), 2.40 (s, 1H); HRMS (ES⁺): Calcd for C₂₂H₂₃N₂O (M⁺+H): 331.1778, Found: 331.1826.

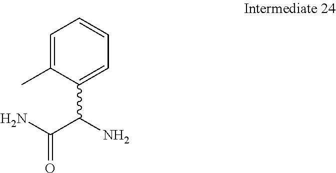

α-Amino-2-methylbenzeneacetamide. A mixture of α-[diphenylmethyl)amino]-2-methylbenzeneacetamide (3.6 g, 10.9 mmol), 10% Pd/C (360 mg), a 1N HCl solution (11 mL, 11 mmol) and MeOH (50 mL) was shaken in a Parr apparatus under a hydrogen atmosphere (pressure ≦50 psi) for 5 h (17 psi taken up). The system was purged with nitrogen and the reaction mixture was filtered through Celite. Solvent was removed in vacuo. The residue was triturated with copious amounts of ether and filtered. Drying in vacuo afforded a white solid (1.05 g, 59% yield): ¹H NMR (DMSO-d₆, 300 MHz): 7.39 (s, 1H), 7.23 (m, 5H), 4.46 (s, 1H) 2.37 (s, 3H), 2.09 (s, 2H); HRMS (ES⁺): Calcd for C₁₉H₁₃NO₂ (M⁺+H): 165.1023, Found: 165.1029.

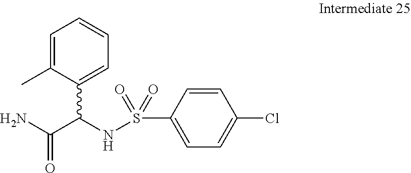

α-[4-Chlorobenzenesulfonylamino]-2-methylbenzeneacetamide. Following the procedure outlined for intermediate 8, this example was prepared from α-amino-2-methylbenzeneacetamide (1.05 g, 6.4 mmol), 4-chlorobenzenesulfonyl chloride (1.49 g, 7.04 mmol), Et₃N (1.95 mL, 14 mmol) were reacted in dioxan (10 mL) to give the title product (creme solid, 1.8 g, 83% yield): 8.41 (d, 1H, J=8), 7.70 (dd, 2H, J=8, 1), 7.53 (dd, 2H, J=8, 1), 7.23 (s, 1H), 7.09 (m, 5H), 5.0 (d, 1H, J=7), 2.28 (s, 3H); MS (ES⁻): 337, 339 (M⁺−H).

### Example 1

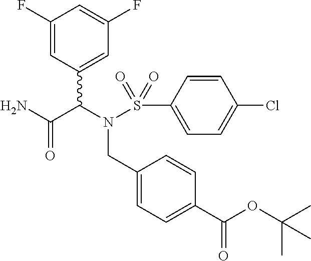

α-[(4-Chlorobenzenesulfonyl)((4-t-butyloxycarbonylphenyl)methyl)amino]-3,5-difluorobenzeneacetamide. α-[4-Chlorobenzenesulfonylamino]-3,5-difluorobenzeneacetamide (300 mg, 0.83 mmol) was dissolved in dry THF (2.5 mL) and the resulting solution was cooled to 0° C. with stirring under a nitrogen atmosphere. Diisopropylazodicarboxylate (420 mg, 409 μL, 2.08 mmol) was added and the reaction mixture was stirred for 15 min. t-Butyl 4-hydroxymethylbenzoate (433 mg, 2.08 mmol) was dissolved in dry THF (2.5 mL) and the resulting solution was cooled to 0° C. with stirring under a nitrogen atmosphere. Triphenylphosphine (545 mg, 2.08 mmol) was added and the reaction mixture was stirred for 15 min. The solution containing the alcohol was added to the other solution in one portion. The reaction mixture was warmed to ambient temperature over 18 h; then it was diluted with EtOAc (50 mL). The organic solution was washed with water (15 mL) four times and with brine (20 mL) twice. Drying over MgSO₄, filtration and concentration of the filtrate in vacuo gave crude product. Column chromatography was performed twice (EtOAc:hexane::1:4, then 1:3 (twice)). The crude product was then triturated six times with a mixture of hexane-ether-MeOH (8:1:1). Drying in vacuo afforded the title product (white solid, 207 mg, 45% yield): ¹H NMR (MeOH-d₄, 300 MHz): 7.81 (d, 2H, J=8), 7.65 (d, 2H, J=8), 7.55 (d, 2H, J=8), 7.10 (d, 2H, J=8), 6.85 (d, 2H, J=8), 6.76 (t, 1H, J=8), 5.82 (s, 1H), 4.83 (m, 4H), 1.57 (s, 9H); HRMS (ES⁺): Calcd for C₂₆H₂₉ClF₂N₃O₅S (M⁺+NH₄): 568.1485, Found: 568.1475.

### Example 2

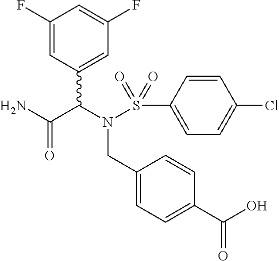

α-[(4-Chlorobenzenesulfonyl)((4-carboxyphenyl)methyl)amino]-3,5-difluorobenzene-acetamide. α-[(4-Chlorobenzenesulfonyl)((4-t-butyloxycarbonylphenyl)methyl)amino]-3,5-difluorobenzeneacetamide (225 mg, 0.41 mmol) was dissolved in DCM (7 mL) and the resulting solution was cooled to 0° C. with stirring under a nitrogen atmosphere. TFA (1.35 mL, 17.5 mmol) was added. The reaction mixture was warmed to room temperature with stirring over 5 h. Solvent was removed in vacuo and the residue was dissolved in EtOAc (20 mL). The organic solution was washed with a 5% NaHSO₄ solution (5 mL) three times and brine (5 mL) twice. Drying over MgSO₄, filtration and concentration of the filtrate in vacuo gave the title product (an off-white solid, 206 mg, 100% yield): ¹H NMR (MeOH-d₄, 300 MHz): 7.9 (s, 1H, concentration dependent), 7.81 (d, 2H, J=8), 7.65 (d, 2H, J=8), 7.55 (d, 2H, J=8), 7.10 (d, 2H, J=8), 6.85 (d, 2H, J=8), 6.76 (t, 1H, J=8), 5.82 (s, 1H), 4.83 (m, 4H); HRMS (ES⁺): Calcd for C₂₂H₁₈ClF₂N₂O₅S (M⁺+H): 495.0593, Found: 495.0605.

### Example 3

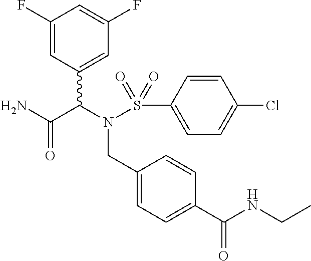

α-[(4-Chlorobenzenesulfonyl)((4-ethylaminocarbonylphenyl)methyl)amino]-3,5-difluorobenzeneacetamide. α-[(4-Chlorobenzenesulfonyl)((4-carboxyphenyl)methyl)amino]-3,5-difluorobenzene-acetamide (98 mg, 0.2 mmol) was dissolved in DMF (300 μL) and CH₃CN (1 mL) with stirring at room temperature. N,N′-Diisopropyl-N-ethylamine (95 μL, 0.55 mmol) was added, followed by benzotriazo-1-yloxytripyrrolidinophosphonium hexafluorophosphate (PyBOP) (103 mg, 0.2 mmol). Stirring was continued for 5 min. Ethylamine (2M in THF, 149 μL, 0.3 mmol) was added and the reaction mixture was stirred for 1.5 h. The reaction mixture was diluted with EtOAc (15 mL) and the solution was washed with a 5% NaHSO₄ solution (5 mL) three times and brine (5 mL) twice). Drying over MgSO₄, filtration and concentration of the filtrate in vacuo gave crude product. Column chromatography was performed (EtOAc:hexane:3:2). The crude product was then triturated with a mixture of hexane-ether-MeOH (8:1:1). Drying in vacuo afforded the title product (white solid, 86.3 mg, 83% yield): ¹H NMR (DMSO-d₆, 300 MHz): 8.32 (t, 1H, J=6), 7.82 (d, 2H, J=8), 7.69 (s, 1H), 7.66 (d, 2H, J=8), 7.55 (d, 2H, J=8), 7.42 (s, 1H), 7.12 (td, 1H, J=8, 1), 7.04 (d, 2H, J=8), 6.76 (dt, 2H, J=8, 1), 5.71 (s, 1H), 4.78 (s, 2H), 3.70 (q, 2H, J=7), 1.10 (t, 3H, J=7); HRMS (ES⁺): Calcd for C₂₄H₂₃ClF₂N₃O₄S (M⁺+H): 522.1066, Found: 522.1049.

Separation of the enantiomers of α-[(4-Chlorobenzenesulfonyl)((4-ethylaminocarbonylphenyl)methyl)amino]-3,5-difluorobenzene-acetamide. Chiral chromatography (Chiralcel OD column (4.6×250 mm, 10 μM), 85% hexane: 15% EtOH at 1.0 mL/min, Shimadzu Model LC-8A high pressure preparative liquid chromatograph (HPLC) gave two enantiomers after removal of solvent in vacuo.

### Example 3a

Low retention time enantiomer (retention time=10.3 min, 27.6 mg, 98.8% ee): ¹H NMR (MeOH-d₄, 300 MHz): 7.81 (d, 2H, J=8), 7.53 (m, 4H), 7.12 (d, 2H, J=8), 6.85 (dt, 2H, J=8, 1), 6.74 (td, 1H, J=8, 1), 5.81 (s, 1H), 4.88 (m, 3H), 4.75 (d, 1H, J=16), 3.38 (q, 2H, J=7), 1.30 (t, 3H, J=7); HRMS (ES⁺): Calcd for C₂₄H₂₃ClF₂N₃O₄S (M⁺+H): 522.1066, Found: 522.1052.

### Example 3b

High retention time enantiomer (retention time=12.3 min, 28.8 mg, 99% ee): ¹H NMR (MeOH-d₄, 300 MHz): 7.81 (d, 2H, J=8), 7.53 (m, 4H), 7.12 (d, 2H, J=8), 6.85 (dt, 2H, J=8, 1), 6.74 (td, 1H, J=8, 1), 5.81 (s, 1H), 4.88 (m, 3H), 4.75 (d, 1H, J=16), 3.38 (q, 2H, J=7), 1.30 (t, 3H, J=7); HRMS (ES⁺): Calcd for C₂₄H₂₃ClF₂N₃O₄S (M⁺+H): 522.1066, Found: 522.1058.

Examples 4-8 were prepared according to the procedures above.

### Example 4

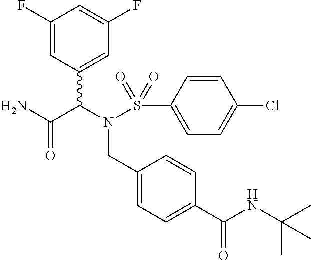

α-[(4-Chlorobenzenesulfonyl)(4-t-butylaminocarbonylphenylmethyl)amino]-3,5-difluorobenzeneacetamide. 56.5 mg (69% yield after chromatography (EtOAc:hexane::2:3)): ¹H NMR (MeOH-d₄, 300 MHz): 7.79 (d, 2H, J=8), 7.51 (m, 5H), 7.08 (d, 2H, J=8), 6.87 (d, 2H, J=8), 6.74 (t, 1H, J=8), 5.80 (s, 1H), 4.78 (m, 4H), 1.44 (s, 9H); HRMS (ES⁺): Calcd for C₂₆H₂₇ClF₂N₃O₄S (M⁺+H): 550.1379, Found: 550.1363.

### Example 5

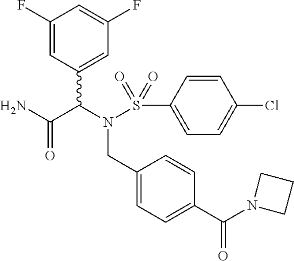

α-[(4-Chlorobenzenesulfonyl)(4-azetidinylcarbonylphenylmethyl)amino]-3,5-difluorobenzene-acetamide. 88.8 mg (82% yield after chromatography (EtOAc:hexane:1: to 65:35)): ¹H NMR (DMSO-d₆, 300 MHz): 7.85 (d, 2H, J=8), 7.68 (s, 1H), 7.67 (d, 2H, J=8), 7.40 (s, 1H), 7.32 (d, 2H, J=8), 7.08 (t, 1H, J=8), 7.05 (d, 2H, J=8), 6.81 (d, 2H, J=8), 5.71 (s, 1H), 4.80 (dd, 2H, J=16,16), 4.20 (t, 2H, J=7), 4.01 (t, 2H, J=7), 2.26 (quintet, 2H, J=7); HRMS (ES⁺): Calcd for C₂₅H₂₃ClF₂N₃O₄S (M⁺+H): 534.1066, Found: 534.1058.

### Example 6

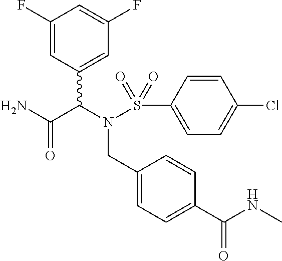

α-[(4-Chlorobenzenesulfonyl)(4-methylaminocarbonylphenylmethyl)amino]-3,5-difluorobenzene-acetamide. 34.3 mg (33% yield after reverse phase HPLC (CH₃CN: H₂O:TFA:30:70:1 to 70:30:1), trituration with ether and drying in vacuo: ¹H NMR (DMSO-d₆, 300 MHz): 7.83 (d, 2H, J=8), 7.66 (d, 2H, J=8), 7.66 (d, 2H, J=8), 7.57 (d, 2H, J=8), 7.39 (s, 1H), 7.08 (t, 1H, J=8), 7.06 (d, 2H, J=8), 6.82 (d, 2H, J=8), 5.71 (s, 1H), 4.79 (s, 2H), 2.75 (d, 3H, J=2); HRMS (ES⁺): Calcd for C₂₃H₂₁ClF₂N₃O₄S (M⁺+H): 508.0909, Found: 508.0906.

### Example 7

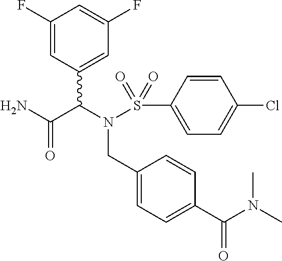

α-[(4-Chlorobenzenesulfonyl)((4-dimethylaminocarbonylphenylmethyl)amino]-3,5-difluorobenzene-acetamide. 82.3 mg (78% yield after chromatography (EtOAc:hexane:2:3 to 45:55): ¹H NMR (DMSO-d₆, 300 MHz): 7.84 (dd, 2H, J=8, 1), 7.69 (m, 2H), 7.68 (dd, 2H, J=8, 1), 7.41 (s, 1H), 7.11 (d, 2H, J=8), 7.07 (d, 2H, J=8), 6.82 (d, 2H, J=8), 5.72 (s, 1H), 4.80 (dd, 2H, J=16,16), 3.23 (s, 6H); HRMS (ES⁺): Calcd for C₂₄H₂₃ClF₂N₃O₄S (M⁺+H): 522.1066, Found: 522.1066.

### Example 8

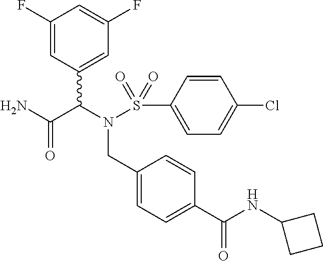

α-[(4-Chlorobenzenesulfonyl)((4-cyclobutylaminocarbonylphenylmethyl)amino]-3,5-difluorobenzeneacetamide. 90.9 mg (82% yield after chromatography (EtOAc:hexane::2:3): ¹H NMR (DMSO-d₆, 300 MHz): 8.42 (d, 1H, J=8), 7.84 (d, 2H, J=8), 7.67 (m, 1H), 7.65 (d, 2H, J=8), 7.41 (s, 1H), 7.15 (t, 1H, J=8), 7.03 (d, 2H, J=8), 7.07 (d, 2H, J=8), 6.82 (d, 2H, J=8), 5.71 (s, 1H), 4.78 (s, 2H), 4.39 (m, 1H), 2.24 (m, 2H), 2.12 (m, 2H), 1.65 (m, 2H); HRMS (ES⁺): Calcd for C₂₆H₂₅ClF₂N₃O₄S (M⁺+H): 548.1222, Found: 548.1232.

Examples 9-16 were prepared according to the procedures above using α-[4-chlorobenzenesulfonylamino]-3,5-difluorobenzeneacetamide, the appropriate alcohol (2.5 equivalents), triphenylphosphine (2.5 equivalents) and diisopropylazodicarboxylate (2.5 equivalents).

### Example 9

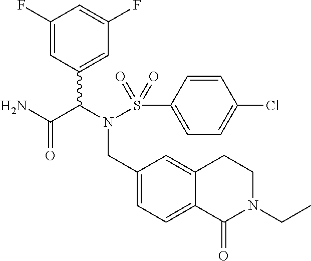

α-[(4-Chlorobenzenesulfonyl)(1-oxo-2-ethyl-1,2,3,4-tetrahydroisoquinolin-6-ylmethyl)amino]-3,5-difluorobenzeneacetamide. 31.1 mg (14% yield, using 2-ethyl-6-hydroxymethyl-2H-1,2,3,4-tetrahydro-isoquinolone, following column chromatography (EtOAc:hexane::6:4 then 7:3), then reverse phase HPLC (CH₃CN:H₂O:TFA::30:70:1 to 70:30:1), then column chromatography (MeOH:CHCl₃::2:98)): ¹H NMR (MeOH-d₄, 300 MHz): 7.82 (d, 2H, J=8), 7.64 (d, 1H, J=8), 7.57 (m, 1H), 7.55 (d, 2H, J=8), 7.03 (d, 1H, J=8), 6.86 (d, 2H, J=8), 6.76 (t, 1H, J=8), 5.83 (s, 1H), 4.87 (d, 1H, J=16), 4.85 (m, 2H), 4.73 (d, 1H, J=16), 3.57 (m, 4H), 2.82 (t, 2H, J=7), 1.20 (t, 3H, J=7); HRMS (ES⁺): Calcd for C₂₆H₂₅ClF₂N₃O₄S (M⁺+H): 548.1222, Found: 548.1212.

Separation of the enantiomers of α-[(4-chlorobenzenesulfonyl)(1-oxo-2-ethyl-1,2,3,4-tetrahydroisoquinolin-6-yl)methylamino]-3,5-difluorobenzene-acetamide.

Chiral chromatography (Chiralcel OD column (5×50 cm, 20 μm), heptane:EtOH::3:1 at 1.0 mL/min, Shimadzu Model LC-8A high pressure preparative liquid chromatograph (HPLC) gave two enantiomers after removal of solvent in vacuo.

### Example 9a

Low retention time enantiomer (retention time=7.3 min, 6.6 mg, 99.3% ee): 7.82 (d, 2H, J=8), 7.64 (d, 1H, J=8), 7.57 (m, 1H), 7.55 (d, 2H, J=8), 7.03 (d, 1H, J=8), 6.86 (d, 2H, J=8), 6.76 (t, 1H, J=8), 5.83 (s, 1H), 4.87 (d, 1H, J=16), 4.85 (m, 2H), 4.73 (d, 1H, J=16), 3.57 (m, 4H), 2.82 (t, 2H, J=7), 1.20 (t, 3H, J=7); HRMS (ES⁺): Calcd for C₂₆H₂₅ClF₂N₃O₄S (M⁺+H): 548.1222, Found: 548.1207.

### Example 9b

High retention time enantiomer (retention time=10.4 min, 6.4 mg, 99.0% ee): 7.82 (d, 2H, J=8), 7.64 (d, 1H, J=8), 7.57 (m, 1H), 7.55 (d, 2H, J=8), 7.03 (d, 1H, J=8), 6.86 (d, 2H, J=8), 6.76 (t, 1H, J=8), 5.83 (s, 1H), 4.87 (d, 1H, J=16), 4.85 (m, 2H), 4.73 (d, 1H, J=16), 3.57 (m, 4H), 2.82 (t, 2H, J=7), 1.20 (t, 3H, J=7); HRMS (ES⁺): Calcd for C₂₆H₂₅ClF₂N₃O₄S (M⁺+H): 548.1222, Found: 548.1247.

### Example 10

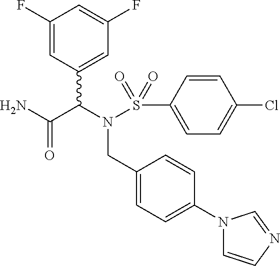

α-[(4-Chlorobenzenesulfonyl)(4-imidazolylphenylmethyl)amino]-3,5-difluorobenzene-acetamide. 49.6 mg (28% yield, using 4-imidazolyl-1-(hydroxymethyl)benzene, following column chromatography (MeOH:CHCl₃::2:98), then trituration with ether-hexanes (1:3), then drying in vacuo): ¹H NMR (MeOH-d₄, 300 MHz): 8.03 (s, 1H), 7.82 (d, 2H, J=8), 7.55 (d, 2H, J=8), 7.50 (m, 1H), 7.47 (s, 1H), 7.28 (d, 2H, J=8), 7.18 (s, 1H), 6.87 (d, 2H, J=8), 6.75 (t, 1H, J=8), 5.84 (s, 1H), 4.80 (d, 1H, J=16), 4.77 (m, 2H), 4.73 (d, 2H, J=16); HRMS (ES⁺): Calcd for C₂₄H₂₀ClF₂N₄O₃S (M⁺+H): 517.0913, Found: 517.0925.

### Example 11

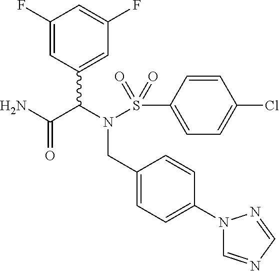

α-[(4-Chlorobenzenesulfonyl)(4-(1,2,4-triazolyl)-phenylmethyl)amino]-3,5-difluorobenzene-acetamide. 107.9 mg (60% yield, using 4-(1,2,4-triazolyl)-1-(hydroxymethyl)benzene, following column chromatography (MeOH:CHCl₃::2:98): ¹H NMR (MeOH-d₄, 300 MHz): 9.00 (s, 1H), 8.13 (s, 1H), 7.82 (d, 2H, J=8), 7.53 (m, 4H), 7.21 (d, 2H, J=8), 6.88 (d, 2H, J=8), 6.80 (t, 1H, J=8), 5.83 (s, 1H), 4.88 (d, 1H, J=16), 4.70 (m, 2H), 4.76 (d, 1H, J=16); HRMS (ES⁺): Calcd for C₂₃H₁₉ClF₂N₅O₃S (M⁺+H): 518.0865, Found: 518.0884.

### Example 12

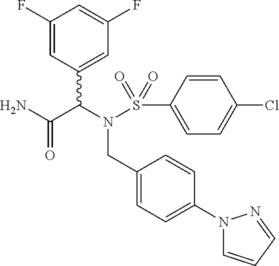

α-[(4-Chlorobenzenesulfonyl)(4-pyrazolylphenylmethyl)amino]-3,5-difluorobenzene-acetamide. 259 mg (30% yield, using 4-pyrazolyl-1-(hydroxymethyl)benzene, following column chromatography (EtOAc:hexane::1:1) then trituration with ether-hexane(1:3), then drying in vacuo: ¹H NMR (MeOH-d₄, 300 MHz): 8.12 (d, 1H, J=1), 7.81 (d, 2H, J=8), 7.68 (d, 1H, J=1), 7.54 (d, 2H, J=8), 7.44 (d, 2H, J=8), 7.15 (d, 2H, J=8), 6.88 (d, 2H, J=8), 6.49 (t, 1H, J=8), 6.50 (d, 1H, J=1), 5.82 (s, 1H), 4.86 (d, 1H, J=16), 4.84 (m, 2H), 4.73 (d, 1H, J=16); HRMS (ES⁺): Calcd for C₂₄H₁₉ClF₂N₄O₃SNa (M⁺+Na): 539.0732, Found: 539.0748.

Separation of the enantiomers of α-[(4-Chlorobenzenesulfonyl)(4-pyrazolylphenylmethyl)amino]-3,5-difluorobenzene-acetamide. Chiral chromatography (Chiralcel AD column (5×50 cm, 20 μm), heptane:EtOH::85:15 at 1.0 mL/min, Shimadzu Model LC-8A high pressure preparative liquid chromatograph (HPLC) gave two enantiomers after removal of solvent in vacuo.

### Example 12a

Low retention time enantiomer (106.4 mg, retention time=21.2 min, 99.2% ee): ¹H NMR (MeOH-d₄, 300 MHz): 8.09 (d, 1H, J=1), 7.79 (d, 2H, J=8), 7.66 (d, 1H, J=1), 7.53 (d, 2H, J=8), 7.42 (d, 2H, J=8), 7.12 (d, 2H, J=8), 6.87 (d, 2H, J=8), 6.72 (t, 1H, J=8), 6.50 (d, 1H, J=1), 5.80 (s, 1H), 4.86 (d, 1H, J=16), 4.84 (m, 2H), 4.73 (d, 1H, J=16); HRMS (ES⁺): Calcd for C₂₄H₂₀ClF₂N₄O₃S (M⁺+H): 517.0913, Found: 517.0906.

### Example 12b

Low retention time enantiomer (106.4 mg, retention time=21.2 min, 99.2% ee): ¹H NMR (MeOH-d₄, 300 MHz): 8.09 (d, 1H, J=1), 7.79 (d, 2H, J=8), 7.66 (d, 1H, J=1), 7.53 (d, 2H, J=8), 7.42 (d, 2H, J=8), 7.12 (d, 2H, J=8), 6.87 (d, 2H, J=8), 6.72 (t, 1H, J=8), 6.50 (d, 1H, J=1), 5.80 (s, 1H), 4.86 (d, 1H, J=16), 4.84 (m, 2H), 4.73 (d, 1H, J=16); HRMS (ES⁺): Calcd for C₂₄H₂₀ClF₂N₄O₃S (M⁺+H): 517.0913, Found: 517.0901.

### Example 13

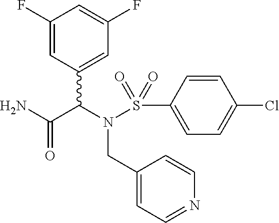

α-[(4-Chlorobenzenesulfonyl)(4-pyridylmethyl)amino]-3,5-difluorobenzene-acetamide. 26.2 mg (34% yield, using 4-(hydroxymethyl)pyridine, following column chromatography (MeOH:CHCl₃::2:98, then EtOAc:hexane:Et₃N::50:50:1): ¹H NMR (MeOH-d₄, 300 MHz): 8.23 (d, 2H, J=8), 7.87 (d, 2H, J=8), 7.59 (d, 2H, J=8), 7.12 (d 2H, J=8), 6.89 (d, 2H, J=8), 6.76 (t, 1H, J=8), 5.83 (s, 1H), 4.84 (m, 4H); HRMS (ES⁺): Calcd for C₂₀H₁₇ClF₂N₃O₃S (M⁺+H): 452.0647, Found: 452.0643.

### Example 14

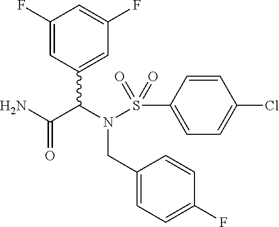

α-[(4-Chlorobenzenesulfonyl)(4-fluorophenylmethyl)amino]-3,5-difluorobenzene-acetamide. 26.2 mg (11% yield, using 4-fluoro-1-(hydroxymethyl)benzene, following column chromatography (MeOH:CHCl₃::0.5::99.5, then EtOAc:hexane::25:75): ¹H NMR (MeOH-d₄, 300 MHz): 7.79 (d, 2H, J=8), 7.55 (d, 2H, J=8), 7.02 (m, 2H), 6.82 (m, 5H), 5.79 (s, 1H), 4.82 (m, 2H), 4.78 (d, 1H, J=16), 4.67 (d, 1H, J=16); HRMS (ES⁺): Calcd for C₂₁H₁₇ClF₃N₂O₃S (M⁺+H): 469.0601, Found: 469.0607.

### Example 15

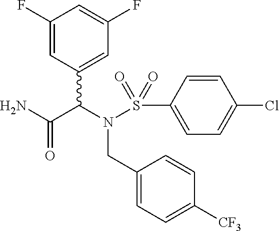

α-[(4-Chlorobenzenesulfonyl)(4-trifluoromethylphenylmethyl)amino]-3,5-difluorobenzene-acetamide. 70.5 mg (49% yield, using 4-trifluoromethyl-1-(hydroxymethyl)benzene, following column chromatography (EtOAc:hexane::1:1, then EtOAc:hexane:Et₃N::75:25:0.5, then EtOAc:hexane:Et₃N::25:75:0.5): ¹H NMR (MeOH-d₄, 300 MHz):7.83 (d, 2H, J=8), 7.57 (d, 2H, J=8), 7.35 (d, 2H, J=8), 7.22 (d, 2H, J=8), 6.84 (d, 2H, J=8), 6.72 (t, 1H, J=8), 5.83 (s, 1H), 4.83 (m, 4H); MS (ES⁺): 519, 521 (M⁺+H).

Separation of enantiomers of α-[(4-Chlorobenzenesulfonyl)(4-trifluoromethylphenyl)methylamino]-3,5-difluorobenzeneacetamide. Chiral chromatography (Chiralcel AD column (5×50 cm, 20 μm), heptane:iPrOH::9:1 at 1.0 mL/min, Shimadzu Model LC-8A high pressure preparative liquid chromatograph (HPLC) gave two enantiomers after removal of solvent in vacuo.

### Example 15a

Low retention time enantiomer (retention time=12.4 min, 25.4 mg, 99.2% ee): ¹H NMR (MeOH-d₄, 300 MHz): 7.82 (d, 2H, J=8), 7.55 (d, 2H, J=8), 7.35 (d, 2H, J=8), 7.23 (d, 2H, J=8), 6.84 (d, 2H, J=8, 6.70 (t, 1H, J=8), 5.83 (s, 1H), 4.84 (d, 1H, J=16), 4.80 (m, 2H), 4.78 (d, 1H, J=16); HRMS (ES⁺): Calcd for C₂₂H₁₇ClF₅N₂O₃S (M⁺+H): 519.0569, Found: 519.0579.

### Example 15b

High retention time enantiomer (retention time=17.2 min, 11.7 mg, 98.7% ee): ¹H NMR (MeOH-d₄, 300 MHz): 7.82 (d, 2H, J=8), 7.55 (d, 2H, J=8), 7.35 (d, 2H, J=8), 7.23 (d, 2H, J=8), 6.84 (d, 2H, J=8, 6.70 (t, 1H, J=8), 5.83 (s, 1H), 4.84 (d, 1H, J=16), 4.80 (m, 2H), 4.78 (d, 1H, J=16); HRMS (ES⁺): Calcd for C₂₂H₁₇ClF₅N₂O₃S (M⁺+H): 519.0569, Found: 519.0561.

### Example 16

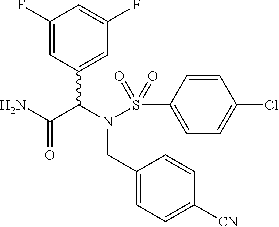

α-[(4-Chlorobenzenesulfonyl)(4-cyanophenylmethyl)amino]-3,5-difluorobenzene-acetamide. 75.5 mg (57% yield, using 4-cyano-1-(hydroxymethyl)benzene, following column chromatography (EtOAc:hexane:Et₃N::25:75:0.5, then EtOAc:hexane:Et₃N::40:60:0.5): ¹H NMR (MeOH-d₄, 300 MHz): 7.84 (d, 2H, J=8), 7.57 (d, 2H, J=8), 7.45 (d, 2H, J=8), 7.23 (d, 2H, J=8), 6.85 (d, 2H, J=8), 6.77 (t, 1H, J=8), 5.82 (s, 1H), 4.90 (d, 1H, J=16), 4.86 (m, 2H), 4.83 (d, 1H, J=16).

Separation of enantiomers of α-[(4-Chlorobenzenesulfonyl)(4-cyanophenylmethyl)amino]-3,5-difluorobenzene-acetamide. Chiral chromatography (Chiralcel AD column (5×50 cm, 20 μm), heptane:iPrOH::4:1 at 1.0 mL/min, Shimadzu Model LC-8A high pressure preparative liquid chromatograph (HPLC) gave two enantiomers after removal of solvent in vacuo.

### Example 16a

Low retention time enantiomer (retention time=10.4 min, 23.9 mg, 98.6% ee): ¹H NMR (MeOH-d₄, 300 MHz): 7.84 (d, 2H, J=8), 7.57 (d, 2H, J=8), 7.45 (d, 2H, J=8), 7.23 (d, 2H, J=8), 6.85 (d, 2H, J=8), 6.77 (t, 1H, J=8), 5.82 (s, 1H), 4.87 (d, 1H, J=16), 4.83 (m, 2H), 4.77 (d, 1H, J=16); HRMS (ES⁺): Calcd for C₂₂H₁₇ClF₂N₃O₃S (M⁺+H): 476.0647, Found: 476.0661.

### Example 16b

High retention time enantiomer (retention time=16.2 min, 21.9 mg, 99.1% ee): ¹H NMR (MeOH-d₄, 300 MHz): 7.84 (d, 2H, J=8), 7.57 (d, 2H, J=8), 7.45 (d, 2H, J=8), 7.23 (d, 2H, J=8), 6.85 (d, 2H, J=8), 6.77 (t, 1H, J=8), 5.82 (s, 1H), 4.87 (d, 1H, J=16), 4.83 (m, 2H), 4.77 (d, 1H, J=16); HRMS (ES⁺): Calcd for C₂₂H₁₇ClF₂N₃O₃S (M⁺+H): 476.0647, Found: 476.0651.

### Example 17a

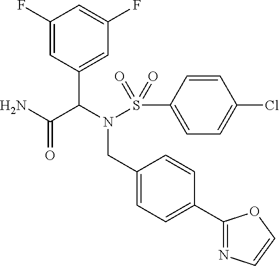

α-[(4-chlorophenylsulfonyl)(4-(oxazol-2-yl)phenylmethyl)amino]-3,5-difluorobenzeneacetamide, enantiomer 1. α-(4-chlorophenylsulfonylamino)-3,5-difluorobenzeneacetamide, low retention time enantiomer (238 mg, 0.66 mmol) was dissolved in DMF (2 mL) and the resulting solution was cooled to 0° C. with stirring under a nitrogen atmosphere. 2-(4-(bromomethyl)-phenyl)oxazole (236 mg, 0.99 mmol) was added next, followed by addition of cesium carbonate (472 mg, 1.45 mmol). The reaction mixture was warmed to room temperature with stirring over 1.5 h. The reaction mixture was diluted with EtOAc (75 mL) and the solution was washed with a saturated NaHCO₃ solution (15 mL) three times, with a 5% LiCl solution (15 mL) three times and with brine (15 mL) twice. Drying over MgSO₄, filtration and concentration of the filtrate in vacuo gave crude product. Column chromatography was performed (MeOH:CHCl₃::0.5:99.5), followed by removal of solvent in vacuo to give the title product (white powder, 29.2 mg, 8.5% yield): ¹H NMR (MeOH-d₄, 300 MHz): 7.94 (s, 1H), 7.82 (d, 2H, J=9), 7.72 (d, 2H, J=9), 7.54 (d, 2H, J=9), 7.25 (s, 1H), 7.16 (d, 2H, J=9), 6.86 (m, 2H, J=7), 6.71 (m, 1H), 5.81 (s, 1H), 4.81 (m, 4H); HRMS (ES⁺): Calcd for C₂₄H₁₉ClF₂N₃O₄S (M⁺+H): 518.0753, Found: 518.0774.

### Example 17b

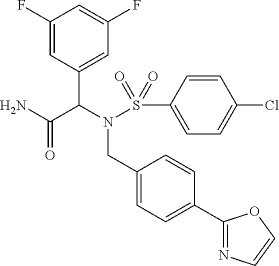

α-[(4-chlorophenylsulfonyl)(4-(oxazol-2-yl)phenylmethyl)amino]-3,5-difluorobenzeneacetamide, enantiomer 2. α-(4-chlorophenylsulfonylamino)-3,5-difluorobenzeneacetamide, high retention time enantiomer (145 mg, 0.40 mmol) was dissolved in DMF (2 mL) and the resulting solution was cooled to 0° C. with stirring under a nitrogen atmosphere. 2-(4-(Bromomethyl)phenyl)oxazole (144 mg, 0.60 mmol) was added next, followed by addition of cesium carbonate (288 mg, 0.88 mmol). The reaction mixture was warmed to room temperature with stirring over 1.5 h. The reaction mixture was diluted with EtOAc (50 mL) and the solution was washed with a saturated NaHCO₃ solution (10 mL) three times, with a 5% LiCl solution (10 mL) three times and with brine (10 mL) twice. Drying over MgSO₄, filtration and concentration of the filtrate in vacuo gave crude product. Column chromatography was performed (MeOH/CHCl₃::0.5:99.5), followed by removal of solvent in vacuo to give the title product (white film, 53.5 mg, 26% yield): ¹H NMR (MeOH-d₄, 300 MHz): 7.94 (s, 1H), 7.82 (d, 2H, J=9), 7.72 (d, 2H, J=9), 7.54 (d, 2H, J=9), 7.25 (s, 1H), 7.16 (d, 2H, J=9), 6.86 (m, 2H, J=7), 6.71 (m, 1H), 5.81 (s, 1H), 4.81 (m, 4H); HRMS (ES⁺): Calcd for C₂₄H₁₉ClF₂N₃O₄S (M⁺+H): 518.0753, Found: 518.0754.

### Example 18

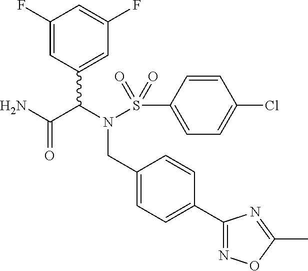

α-[(4-chlorophenylsulfonyl)(4-(4-(5-methyl-1,2,4-oxadiazol-3-yl)phenylmethyl)amino]-3,5-difluorobenzeneacetamide. α-(4-Chlorophenylsulfonylamino)-3,5-difluorobenzeneacetamide (300 mg, 0.83 mmol) was dissolved in DMF (5 mL). To this mixture was added 3-(4-(bromomethyl)phenyl)-5-methyl-1,2,4-oxadiazole (340 mg, 1.08 mmol), and Cs₂CO₃ (810 mg, 2.5 mmol). The reaction mixture was stirred at room temperature for 6 h.

The reaction mixture was then poured onto ethyl acetate (30 mL). The organic mixture was washed with saturated Na₂CO₃ (10 mL) twice, then with brine (10 mL) twice. The organic solution was dried over MgSO₄ and filtered; the filtrate was concentrated in vacuo. The crude residue was purified by medium pressure liquid chromatography (MPLC) using the Biotage Horizon 2.0 system (EtOAc:hexanes::1:4 to 4:1, total solvent volume=2 L) to give the title product as a white solid (117 mg, 26% yield): MS (ES⁺): 333 (M⁺+H).

Separation of enantiomers of α-[(4-chlorophenylsulfonyl)(4-(4-(5-methyl-1,2,4-oxadiazol-3-yl)phenylmethyl)amino]-3,5-difluorobenzeneacetamide. Chiral chromatography (117 mg) (Chiralcel OD column (5×50 cm, 20 μm), heptane:EtOH::9:1 at 70.0 mL/min, Shimadzu Model LC-8A high pressure preparative liquid chromatograph (HPLC) gave two enantiomers after removal of solvent in vacuo.

### Example 18a

Low Retention time enantiomer (retention time=18.5 min, 28 mg, 98.4% ee): ¹H-NMR (DMSO-d₆, 500 MHz): 7.85 (d, 2H, J=9), 7.75-7.60 (m, 5H), 7.38 (s, 1H), 7.16 (d, 2H, J=8), 7.03 (t, 1H, J=6), 6.83 (d, 2H, J=6), 5.73 (s, 1H), 4.83 (s, 2H), 2.65 (s, 3H); MS (ES⁺): 533, 535 (M⁺+H).

### Example 18b

High Retention time enantiomer (retention time=23.6 min, 37 mg, 98.8% ee): ¹H-NMR (DMSO-d₆, 500 MHz): 7.85 (d, 2H, J=9), 7.75-7.60 (m, 5H), 7.38 (s, 1H), 7.16 (d, 2H, J=8), 7.03 (t, 1H, J=6), 6.83 (d, 2H, J=6), 5.73 (s, 1H), 4.83 (s, 2H), 2.65 (s, 3H); HRMS (ES⁺): Calcd for C₂₄H₂₀ClF₂N₄O₄S (M⁺+H): 533.0862, Found: 533.0836.

### Example 19

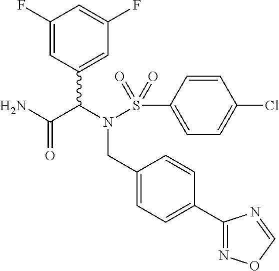

α-[(4-chlorophenylsulfonyl)(4-(4-(1,2,4-oxadiazol-3-yl)phenylmethyl)amino]-3,5-difluorobenzeneacetamide. α-(4-Chlorophenylsulfonylamino)-3,5-difluorobenzeneacetamide (200 mg, 0.56 mmol) was dissolved in DMF (2 mL). To this mixture was added 3-(4-(bromomethyl)phenyl)-1,2,4-oxadiazole (200 mg, 0.84 mmol), and Cs₂CO₃ (275 mg, 0.84 mmol). The reaction mixture was stirred at room temperature for 3 h. The reaction mixture was then poured onto ethyl acetate (10 mL). The organic mixture was washed with saturated Na₂CO₃ (1 mL) twice, then with brine (1 mL) twice. The organic solution was dried over MgSO₄ and filtered; the filtrate was concentrated in vacuo. The crude residue was purified by medium pressure liquid chromatography (MPLC) using the Biotage Horizon 2.0 system (DCM:acetone:hexanes::3:1:6) to give the title product as a white solid (181 mg, 26% yield): MS (ES⁺): 519, 521 (M⁺+H).

Separation of Enantimors of α-[(4-chlorophenylsulfonyl)(4-(4-(1,2,4-oxadiazol-3-yl)phenylmethyl)amino]-3,5-difluorobenzeneacetamide. Chiral chromatography (181 mg) (Chiralcel OD column (5×50 cm, 20 μm), heptane:EtOH::9:1 at 70.0 mL/min, Shimadzu Model LC-8A high pressure preparative liquid chromatograph (HPLC) gave two enantiomers after removal of solvent in vacuo.

### Example 19a

Low Retention time enantiomer (retention time=22 min, 27 mg, 98.8% ee): ¹H-NMR (DMSO-d₆, 500 MHz): 9.66 (s, 1H), 7.84 (d, 2H, J=9), 7.76 (d, 2H, J=8), 7.6-7.7 (m, 3H), 7.38 (s, 1H), 7.2 (d, 2H, J=9) 7.10-7.00 (m, 1H), 6.84 (d, 2H, J=8), 5.73 (s, 1H), 4.84 (s, 2H); MS (ES⁺): 519, 521 (M⁺+H).

### Example 19b

High Retention time enantiomer (retention time=30 min, 25 mg, 99.0% ee): ¹H-NMR (DMSO-d₆, 500 MHz): 9.66 (s, 1H), 7.84 (d, 2H, J=9), 7.76 (d, 2H, J=8), 7.6-7.7 (m, 3H), 7.38 (s, 1H), 7.2 (d, 2H, J=9) 7.10-7.00 (m, 1H), 6.84 (d, 2H, J=8), 5.73 (s, 1H), 4.84 (s, 2H); MS (ES⁺): 519, 521 (M⁺+H).

Examples 20-26 were prepared according to the procedures above.

### Example 20

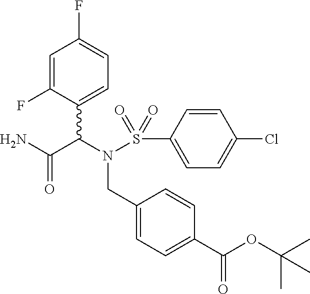

α-[(4-Chlorobenzenesulfonyl)((4-t-butyloxycarbonylphenyl)methyl)amino]-2,4-difluorobenzeneacetamide. 701 mg (61% yield after flash chromatography (EtOAc:hexanes::1:4 to 2:3)): ¹H-NMR (DMSO-d₆, 300 MHz): 7.79 (d, 2H, J=9), 7.62 (d, 2H, J=9), 7.53 (d, 2H, J=9), 7.30 (m, 1H), 7.02 (d, 2H, J=9), 6.82 (t, 1H, J=9), 6.67 (t, 1H, J=9), 6.02 (s, 1H), 4.86 (d, 1H, J=16), 4.85 (m, 2H, concentration dependent), 4.81 (d, 1H, J=16), 1.57 (s, 9H); HRMS (ES⁺): Calcd for C₂₆H₂₆ClF₂N₂O₅S (M⁺+H): 551.1219, Found: 551.1232.

### Example 21

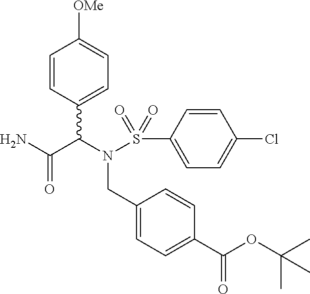

α-[(4-Chlorobenzenesulfonyl)((4-t-butyloxycarbonylphenyl)methyl)amino]-4-methoxybenzeneacetamide. 377 mg (32% yield after flash chromatography (EtOAc:hexanes:: 3:7 to 1:1) then trituration with ether:hexanes::5:95)): ¹H-NMR (MeOH-d₄, 300 MHz): 7.80 (d, 2H, J=9), 7.62 (d, 2H, J=9), 7.58 (s, 1H), 7.55 (d, 2H, J=9), 7.20 (s, 1H), 7.13 (d, 2H, J=9), 6.97 (d, 2H, J=9), 6.78 (d, 2H, J=9), 5.65 (s, 1H), 4.68 (s, 2H), 3.65 (s, 3H), 1.51 (s, 9H); HRMS (ES⁺): Calcd for C₂₇H₃₀ClN₂O₆S (M⁺+H): 545.1513, Found: 545.1501.

### Example 22

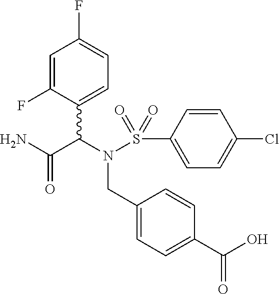

α-[(4-Chlorobenzenesulfonyl)((4-carboxyphenyl)methyl)amino]-2,4-difluorobenzeneacetamide. 616 mg (98% yield after trituration with ether:hexanes::5:95): ¹H-NMR (DMSO-d₆, 300 MHz): 7.85 (d, 2H, J=9), 7.60 (m, 4H), 7.45 (s, 1H), 7.33 (m, 1H), 7.05 (d, 2H, J=9), 6.89 (m, 2H), 5.89 (s, 1H), 4.80 (d, 1H, J=16), 4.66 (d, 1H, J=16), 4.85 (m, 2H, concentration dependent); HRMS (ES⁺): Calcd for C₂₂H₁₈ClF₂N₂O₅S (M⁺+H): 495.0593, Found: 495.0585.

### Example 23

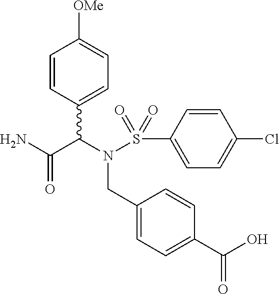

α-[(4-Chlorobenzenesulfonyl)((4-carboxyphenyl)methyl)amino]-4-methoxybenzeneacetamide. 41.5 mg (93% yield after trituration with ether:hexanes::5:95): ¹H-NMR (DMSO-d₆, 300 MHz): 12.75 (s, 1H), 7.81 (d, 2H, J=9), 7.61 (m, 5H), 7.20 (s, 1H), 7.14 (d, 2H, J=9), 6.98 (d, 2H, J=9), 6.78 (d, 2H, J=9), 5.65 (s, 1H), 4.69 (s, 2H), 3.65 (s, 3H); HRMS (ES⁻): Calcd for C₂₃H₂₀ClN₂O₆S (M⁺−H): 487.0731, Found: 487.0747.

### Example 24

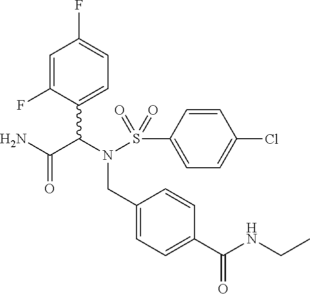

α-[(4-Chlorobenzenesulfonyl)((4-ethylaminocarbonylphenyl)methyl)amino]-2,4-difluorobenzene-acetamide. 88.3 mg (84% yield after chromatography (EtOAc:hexane::1:1 to 35:65): ¹H NMR (DMSO-d₆, 300 MHz): 8.32 (t, 1H, J=6), 7.82 (d, 2H, J=8), 7.69 (s, 1H), 7.66 (d, 2H, J=8), 7.55 (d, 2H, J=8), 7.37 (s, 1H), 7.25 (m, 1H), 6.93 (m, 4H), 5.88 (s, 1H), 4.75 (d, 1H, J=16), 4.65 (d, 1H, J=16), 3.25 (q, 2H, J=7), 1.10 (t, 3H, J=7); HRMS (ES⁺): Calcd for C₂₄H₂₃ClF₂N₃O₄S (M⁺+H): 522.1066, Found: 522.1085.

### Example 25

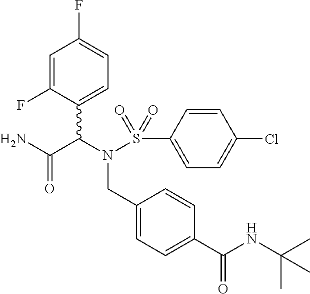

α-[(4-Chlorobenzenesulfonyl)(4-t-butylaminocarbonylphenylmethyl)amino]-2,4-difluorobenzene-acetamide. 87 mg (78% yield after chromatography (EtOAc:hexane::45:55): ¹H NMR (DMSO-d₆, 300 MHz): 7.80 (d, 2H, J=8), 7.75 (s, 1H), 7.64 (d, 2H, J=8), 7.51 (m, 3H), 7.35 (s, 1H), 7.27 (m, 1H), 7.08 (m, 1H), 6.97 (s, 1H), 6.93 (d, 2H, J=8), 5.89 (s, 1H), 4.62 (d, 1H, J=16), 4.54 (d, 1H, J=16), 1.35 (s, 9H); HRMS (ES⁺): Calcd for C₂₆H₂₇ClF₂N₃O₄S (M⁺+H): 550.1379, Found: 550.1380.

### Example 26

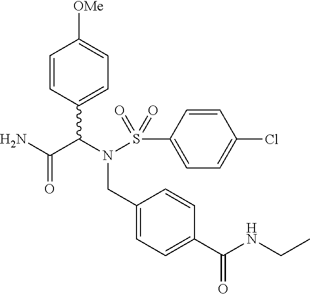

α-[(4-Chlorobenzenesulfonyl)((4-ethylaminocarbonylphenyl)methyl)amino]-4-methoxybenzene-acetamide. 51.8 mg (49% yield after chromatography (EtOAc:hexane::65:35 to EtOAc:hexane:Et₃N::75:25:0.5): ¹H NMR (DMSO-d₆, 300 MHz): 8.29 (t, 1H, J=6), 7.79 (d, 2H, J=9), 7.64 (d, 2H, J=9), 7.55 (s, 1H), 7.52 (d, 2H, J=9), 7.19 (s, 1H), 7.15 (d, 2H, J=9), 6.89 (d, 2H, J=9), 6.81 (d, 2H, J=9), 5.64 (s, 1H), 4.65 (d, 2H), 3.67 (s, 3H), 3.24 (m, 2H), 1.09 (t, 3H, J=7); HRMS (ES⁺): Calcd for C₂₅H₂₇ClN₃O₅S (M⁺+H): 516.1360, Found: 516.1358.

### Example 27

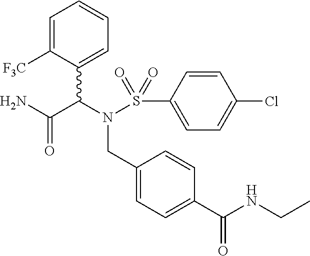

α-[(4-Chlorobenzenesulfonyl)((4-ethylaminocarbonylphenyl)methyl)amino]-2-trifluoromethylbenzene-acetamide. α-[4-Chlorobenzenesulfonylamino]-2-trifluoromethylbenzeneacetamide (164 mg, 0.42 mmol), Cs₂CO₃ (408 mg, 1.25 mmol), KI (83 mg, 0.5 mmol), 4-chloromethylbenzoic acid, ethyl amide (99 mg, 0.5 mmol) and DMF (2 mL) were stirred at room temperature for 18 h. The reaction mixture was diluted with EtOAc (25 mL). The resulting mixture was washed twice with water (8 mL), twice with a saturated NaHCO₃ solution, three times with a 5% LiCl solution, then twice with brine. The organic solution was dried over MgSO₄ and filtered. Solvent was concentrated in vacuo. Flash chromatography (EtOAc:hexanes::3:2) and removal of solvent in vacuo afforded the title product (35.4 mg, 15% yield): ¹H NMR (CDCl₃, 300 MHz): 7.71 (d, 2H, J=9), 7.55 (m 2H), 7.46 (d, 2H, J=9), 7.41 (d, 2H, J=9), 7.33 (m, 2H), 7.04 (d, 2H, J=9), 5.99 (s, 1H), 5.95 (m, 1H), 5.57 (s, 1H), 5.39 (s, 1H), 4.67 (d, 1H, J=16), 4.58 (d, 1H, J=16), 3.45 (m, 2H), 1.22 (t, 3H, J=7); MS (ES⁺): 554, 556 (M⁺+H).

Separation of the enantiomers of α-[(4-Chlorobenzenesulfonyl)((4-ethylaminocarbonylphenyl)methyl)amino]-2-trifluoromethylbenzene-acetamide. Chiral chromatography (Chiralcel AD column (5×50 cm, 20 μm), hexane:EtOH::85:15 at 70.0 mL/min, Shimadzu Model LC-8A high pressure preparative liquid chromatograph (HPLC) gave two enantiomers after removal of solvent in vacuo.

### Example 27a

Low retention time enantiomer (retention time=20 min, 11.6 mg, 98.8% ee): ¹H NMR (CDCl₃, 300 MHz): 7.71 (d, 2H, J=9), 7.55 (m 2H), 7.46 (d, 2H, J=9), 7.41 (d, 2H, J=9), 7.33 (m, 2H), 7.04 (d, 2H, J=9), 5.99 (s, 1H), 5.95 (m, 1H), 5.57 (s, 1H), 5.39 (s, 1H), 4.67 (d, 1H, J=16), 4.58 (d, 1H, J=16), 3.45 (m, 2H), 1.22 (t, 3H, J=7); HRMS (ES⁺): Calcd for C₂₅H₂₄ClF₃N₃O₄S (M⁺+H): 554.1128, Found: 554.1130.

### Example 27b

High retention time enantiomer (retention time=25 min, 13.7 mg, 99% ee): ¹H NMR (CDCl₃, 300 MHz): 7.71 (d, 2H, J=9), 7.55 (m 2H), 7.46 (d, 2H, J=9), 7.41 (d, 2H, J=9), 7.33 (m, 2H), 7.04 (d, 2H, J=9), 5.99 (s, 1H), 5.95 (m, 1H), 5.57 (s, 1H), 5.39 (s, 1H), 4.67 (d, 1H, J=16), 4.58 (d, 1H, J=16), 3.45 (m, 2H), 1.22 (t, 3H, J=7); HRMS (ES⁺): Calcd for C₂₅H₂₄ClF₃N₃O₄S (M⁺+H): 554.1128, Found: 554.1122.

### Example 28

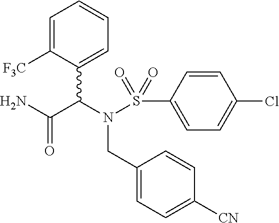

α-[(4-Chlorobenzenesulfonyl)(4-cyanophenylmethyl)amino]-2-trifluoromethyl-benzeneacetamide. Following the procedures above, 318 mg (49% yield, using 4-cyano-1-(hydroxymethyl)benzene, following column chromatography (EtOAc:hexane::4:6): ¹H NMR (CDCl₃, 300 MHz): 7.75 (d, 2H, J=9), 7.65 (m, 1H), 7.53 (m, 2H), 7.45 (d, 2H, J=9), 7.34 (m, 1H), 7.31 (d, 2H, J=9), 7.07 (d, 2H, J=9), 6.10 (s, 1H), 5.46 (s, 1H), 5.45 (s, 1H), 4.70 (d, 1H, J=16), 4.61 (d, 1H, J=16); HRMS (ES⁺): Calcd for C₂₃H₁₈ClF₃N₃O₃S (M⁺+H): 508.0700, Found: 508.0700.

### Example 29

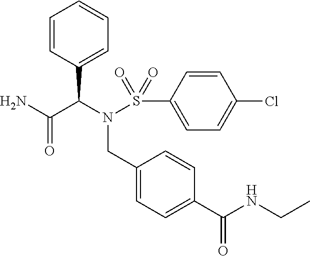

(R)-α-[(4-Chlorobenzenesulfonyl)((4-ethylaminocarbonylphenyl)methyl)amino]-2,4-benzene-acetamide. A mixture of (R)-α-[4-chlorobenzenesulfonylamino]-benzeneacetamide (400 mg, 1.23 mmol), 4-chloromethylbenzoic acid, ethyl amide (365 mg, 1.85 mmol), Cs₂CO₃ (883 mg, 2.7 mmol), KI (204 mg, 1.23 mmol) in DMF (7.5 mL) was stirred at room temperature under a nitrogen atmosphere for 18 h. The reaction mixture was diluted with EtOAc (70 mL) and washed with a saturated NaHCO₃ solution (10 mL) twice, a 5% LiCl solution (10 mL) twice and brine (10 mL) twice. The organic solution was dried over MgSO₄ and filtered. Solvent was removed in vacuo. Column chromatography on the residue (EtOAc:hexane:Et₃N:60:40:0.5) and removal of solvent in vacuo gave the title product (578 mg, 96% yield): ¹H NMR (CDCl₃, 300 MHz): 7.69 (d, 2H, J=8), 7.44 (d, 2H, J=8), 7.40 (d, 2H, J=8), 7.24 (m, 5H), 7.00 (d, 2H, J=8), 5.95 (s, 1H), 5.66 (s, 2H), 5.35 (s, 1H), 4.52 (s, 2H), 3.47 (m, 2H), 1.10 (t, 3H, J=7); HRMS (ES⁺): Calcd for C₂₄H₂₅ClN₃O₄S (M⁺+H): 486.1252, Found: 486.1256.

### Example 30

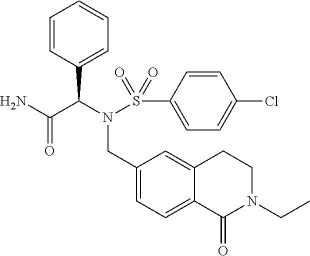

(R)-α-[(4-Chlorobenzenesulfonyl)(1-oxo-2-ethyl-1,2,3,4-tetrahydroisoquinolin-6-ylmethyl)amino]-benzeneacetamide. A mixture of (R)-α-[4-chlorobenzenesulfonylamino]-benzeneacetamide (208 mg, 0.64 mmol), 2-ethyl-5-methanesulfonyloxymethyl-2H-1,2,3,4-tetrahydroisoquinolone (218 mg, 0.77 mmol), Cs₂CO₃ (459 mg, 1.4 mmol) in DMF (5 mL) was stirred at room temperature under a nitrogen atmosphere for 18 h. The reaction mixture was diluted with EtOAc (50 mL) and washed with a saturated NaHCO₃ solution (10 mL) twice, a 5% LiCl solution (10 mL) twice and brine (10 mL) twice. The organic solution was dried over MgSO₄ and filtered. Solvent was removed in vacuo. Column chromatography on the residue (MeOH:CHCl₃::1:99) and removal of solvent in vacuo gave the title product (39 mg, 12% yield): ¹H NMR (CDCl₃, 300 MHz): 7.78 (d, 1H, J=9), 7.69 (d, 2H, J=9), 7.41 (d, 2H, J=9), 7.25 (m, 5H), 6.87 (d, 1H, J=9), 6.70 (s, 1H), 5.68 (s, 1H), 5.65 (s, 1H), 5.41 (s, 1H), 4.56 (d, 1H, J=16), 4.47 (d, 1H, J=16), 3.57 (q, 2H, J=7), 3.44 (t, 2H, J=7), 2.74 (m, 2H), 1.18 (t, 3H, J=7); HRMS (ES⁺): Calcd for C₂₆H₂₂ClN₃O₄S (M⁺+H): 512.1409, Found: 512.1392.

### Example 31

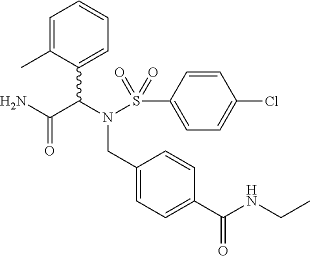

α-[(4-Chlorobenzenesulfonyl)((4-ethylaminocarbonylphenyl)methyl)amino]-2-methylbenzene-acetamide. Following the procedures above and column chromatography (EtOAc) and removal of solvent in vacuo afforded the title product (white solid, 340 mg, 46% yield): ¹H NMR (CDCl₃, 300 MHz): 7.74 (d, 2H, J=9), 7.42 (m, 4H), 7.16 (m, 6H), 6.35 (m, 2H), 6.05 (m, 1H), 4.60 (d, 1H, J=12), 4.40 (d, 1H, J=12), 3.45 (m, 2H), 2.27, 2.21 (2s, 3H); MS (ES⁺): 500, 502 (M⁺+H).

Separation of the enantiomers of α-[(4-Chlorobenzenesulfonyl)((4-ethylaminocarbonylphenyl)methyl)amino]-2-methylbenzene-acetamide. Chiral chromatography (Chiralcel AD column (5×50 cm, 20 μm), hexane:EtOH::85:15 at 70.0 mL/min, Shimadzu Model LC-8A high pressure preparative liquid chromatograph (HPLC) gave two enantiomers after removal of solvent in vacuo.

### Example 31a

Low retention time enantiomer (19 mg, retention time=51 min, 99% ee): ¹H NMR (CDCl₃, 300 MHz): 7.85 (d, 2H, J=9), 7.56 (d, 2H, J=9), 7.45 (d, 2H, J=9), 7.25 (m, 1H), 7.08 (m, 2H), 6.91 (d, 3H, J=9), 5.92 (s, 1H), 4.71 (d, 1H, J=17), 4.59 (d, 1H, J=17), 3.36 (q, 2H, J=7), 2.32 (s, 3H), 1.19 (t, 3H, J=7); HRMS (ES⁺): Calcd for C₂₅H₂₇ClN₃O₄S (M⁺+H): 500.1411, Found: 500.1395.

### Example 31b

High retention time enantiomer (10 mg, retention time=67 min, 98.9% ee): ¹H NMR (CDCl₃, 300 MHz): 7.85 (d, 2H, J=9), 7.56 (d, 2H, J=9), 7.45 (d, 2H, J=9), 7.25 (m, 1H), 7.08 (m, 2H), 6.91 (d, 3H, J=9), 5.92 (s, 1H), 4.71 (d, 1H, J=17), 4.59 (d, 1H, J=17), 3.36 (q, 2H, J=7), 2.32 (s, 3H), 1.19 (t, 3H, J=7); HRMS (ES⁺): Calcd for C₂₅H₂₇ClN₃O₄S (M⁺+H): 500.1411, Found: 500.1409.

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

We claim:

1. A compound of Formula I 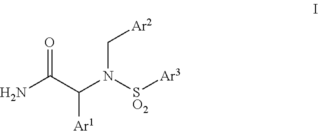
where
Ar¹ is phenyl substituted with 0-5 substituents selected from the group consisting of halo, C₁₋₂haloalkyl, cyano, C₁₋₆alkyl, and C₁₋₆alkoxy;
Ar² is phenyl or pyridinyl, and is substituted with 0-5 substituents selected from the group consisting of halo, C₁₋₂haloalkyl, cyano, C₁₋₆alkyl, C₁₋₆alkoxy, CO₂R¹, CON(R¹)(R¹), CON(R²)(R³), and Ar⁴;
or Ar² is 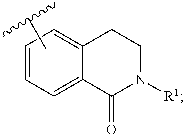
Ar^{a} is 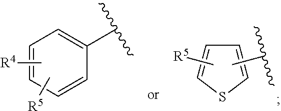
Ar⁴ is a heteroaryl moiety selected from the group consisting of imidazolyl, pyrazolyl, oxadiazolyl, oxazolyl, and triazolyl, and is substituted with 0-2 C₁₋₆alkyl;
R¹ is independently hydrogen, C₁₋₆alkyl, C₃₋₇cycloalkyl, or (C₁₋₄alkoxy)C₁₋₄alkyl;
R² and R³ taken together are CH₂CH₂CH₂, CH₂CH₂CH₂CH₂, CH₂CH₂CH₂CH₂CH₂, CH₂CH₂CH(OH)CH₂CH₂, CH₂CH₂OCH₂CH₂, CH₂CH₂SCH₂CH₂, or CH₂CH₂N(CH₃)CH₂CH₂;
R⁴ is halogen; and
R⁵ is hydrogen or halogen;
or a pharmaceutically acceptable salt thereof.

2. A compound of claim 1 where
Ar¹ is phenyl, dihalophenyl, C₁₋₃alkylphenyl, C₁₋₂haloalkylphenyl, or C₁₋₃alkoxyphenyl;
Ar² is phenyl substituted with 1 substituent selected from the group consisting of halo, C₁₋₂haloalkyl, cyano, CO₂R¹, CON(R¹)(R¹), CON(R²)(R³), and Ar⁴;
or Ar² is pyridinyl or 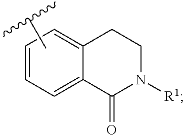
Ar³ is halophenyl;
Ar⁴ is imidazolyl, pyrazolyl, oxazolyl, triazolyl, or oxadiazolyl, and is substituted with 0-1 C₁₋₃alkyl;
R¹ is independently hydrogen, C₁₋₃alkyl, or C₃₋₇cycloalkyl; and
R² and R³ taken together is CH₂CH₂CH₂;
or a pharmaceutically acceptable salt thereof.

3. A compound of claim 2 where
Ar¹ is phenyl, difluorophenyl methylphenyl, trifluoromethylphenyl, or methoxyphenyl;
Ar² is fluorophenyl, trifluoromethylphenyl, cyanophenyl, (alkoxycarbonyl)phenyl, (carboxy)phenyl, (N-methylaminocarbonyl)phenyl, (N-ethylaminocarbonyl)phenyl, (N-t-butylaminocarbonyl)phenyl, (cyclobutylaminocarbonyl)phenyl, (N,N-dimethylaminocarbonyl)phenyl, (azetdinylcarbonyl)phenyl, (pyrazolyl)phenyl, (imidazolyl)phenyl, (triazolyl)phenyl, (oxazolyl)phenyl, (oxadiazolyl)phenyl, (methyloxadiazolyl)phenyl, pyridinyl, or (N-ethyloxotetrahydroisoquinolinyl; and
Ar^{a} is chlorophenyl;
or a pharmaceutically acceptable salt thereof.

4. A compound of claim 1 where Ar¹ is phenyl, halophenyl, dihalophenyl, methylphenyl, trifluoromethylphenyl, or methoxyphenyl and where halo is chloro or fluoro.

5. A compound of claim 1 where Ar² is phenyl substituted with 1 substituent selected from the group consisting of cyano, CO₂R¹, CON(R¹)(R¹), and CON(R²)(R³).

6. A compound of claim 1 where Ar² is phenyl substituted with 1 Ar⁴.

7. A compound of claim 1 where Ar² is

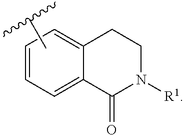

8. A compound of claim 7 where Ar² is

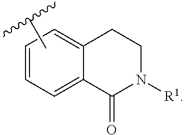

9. A compound of claim 1 where Ar^{a} is 4-chlorophenyl.

10. A compound of claim 1 where Ar⁴ is imidazolyl, pyrazolyl, oxazolyl, oxadiazolyl, triazolyl, methylimidazolyl, methylpyrazolyl, methyloxadiazolyl, or methyltriazolyl.

11. A compound of claim 1 according to formula Ia.

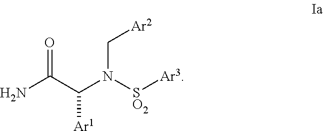

12. A compound of claim 1 selected from the group consisting of
α-[(4-Chlorobenzenesulfonyl)((4-t-butyloxycarbonylphenyl)methyl)amino]-3,5-difluorobenzeneacetamide;
α-[(4-Chlorobenzenesulfonyl)((4-carboxyphenyl)methyl)amino]-3,5-difluorobenzene-acetamide;
α-[(4-Chlorobenzenesulfonyl)((4-ethylaminocarbonylphenyl)methyl)amino]-3,5-difluorobenzeneacetamide;
α-[(4-Chlorobenzenesulfonyl)(4-t-butylaminocarbonylphenylmethyl)amino]-3,5-difluorobenzeneacetamide;
α-[(4-Chlorobenzenesulfonyl)(4-azetidinylcarbonylphenylmethyl)amino]-3,5-difluorobenzene-acetamide;
α-[(4-Chlorobenzenesulfonyl)(4-methylaminocarbonylphenylmethyl)amino]-3,5-difluorobenzene-acetamide;
α-[(4-Chlorobenzenesulfonyl)((4-dimethylaminocarbonylphenylmethyl)amino]-3,5-difluorobenzene-acetamide;
α-[(4-Chlorobenzenesulfonyl)((4-cyclobutylaminocarbonylphenylmethyl)amino]-3,5-difluorobenzeneacetamide;
α-[(4-Chlorobenzenesulfonyl)(1-oxo-2-ethyl-1,2,3,4-tetrahydroisoquinolin-6-ylmethyl)amino]-3,5-difluorobenzeneacetamide;
α-[(4-Chlorobenzenesulfonyl)(4-imidazolylphenylmethyl)amino]-3,5-difluorobenzene-acetamide;
α-[(4-Chlorobenzenesulfonyl)(4-(1,2,4-triazolyl)phenylmethyl)amino]-3,5-difluorobenzene-acetamide;
α-[(4-Chlorobenzenesulfonyl)(4-pyrazolylphenylmethyl)amino]-3,5-difluorobenzene-acetamide;
α-[(4-Chlorobenzenesulfonyl)(4-pyridylmethyl)amino]-3,5-difluorobenzene-acetamide;
α-[(4-Chlorobenzenesulfonyl)(4-fluorophenylmethyl)amino]-3,5-difluorobenzene-acetamide;
α-[(4-Chlorobenzenesulfonyl)(4-trifluoromethylphenylmethyl)amino]-3,5-difluorobenzene-acetamide;
α-[(4-Chlorobenzenesulfonyl)(4-cyanophenylmethyl)amino]-3,5-difluorobenzene-acetamide;
α-[(4-chlorophenylsulfonyl)(4-(oxazol-2-yl)phenylmethyl)amino]-3,5-difluorobenzeneacetamide;
α-[(4-chlorophenylsulfonyl)(4-(4-(5-methyl-1,2,4-oxadiazol-3-yl)phenylmethyl)amino]-3,5-difluorobenzeneacetamide;
α-[(4-chlorophenylsulfonyl)(4-(4-(1,2,4-oxadiazol-3-yl)phenylmethyl)amino]-3,5-difluorobenzeneacetamide;
α-[(4-Chlorobenzenesulfonyl)((4-butyloxycarbonylphenyl)methyl)amino]-2,4-difluorobenzeneacetamide;
α-[(4-Chlorobenzenesulfonyl)((4-butyloxycarbonylphenyl)methyl)amino]-4-methoxybenzeneacetamide;
α-[(4-Chlorobenzenesulfonyl)((4-carboxyphenyl)methyl)amino]-2,4-difluorobenzeneacetamide;
α-[(4-Chlorobenzenesulfonyl)((4-carboxyphenyl)methyl)amino]-4-methoxybenzeneacetamide;
α-[(4-Chlorobenzenesulfonyl)((4-ethylaminocarbonylphenyl)methyl)amino]-2,4-difluorobenzene-acetamide;
α-[(4-Chlorobenzenesulfonyl)((4-butylaminocarbonylphenylmethyl)amino]-2,4-difluorobenzene-acetamide;
α-[(4-Chlorobenzenesulfonyl)((4-ethylaminocarbonylphenyl)methyl)amino]-4-methoxybenzene-acetamide;
α-[(4-Chlorobenzenesulfonyl)((4-ethylaminocarbonylphenyl)methyl)amino]-2-trifluoromethylbenzene-acetamide;
α-[(4-Chlorobenzenesulfonyl)(4-cyanophenylmethyl)amino]-2-trifluoromethyl-benzeneacetamide;
(R)-α-[(4-Chlorobenzenesulfonyl)((4-ethylaminocarbonylphenyl)methyl)amino]-2,4-benzene-acetamide;
(R)-α-[(4-Chlorobenzenesulfonyl)(1-oxo-2-ethyl-1,2,3,4-tetrahydroisoquinolin-6-ylmethyl)amino]-benzeneacetamide; and
α-[(4-Chlorobenzenesulfonyl)((4-ethylaminocarbonylphenyl)methyl)amino]-2-methylbenzene-acetamide;
or a pharmaceutically acceptable salt thereof.

13. A pharmaceutical composition comprising a therapeutically effective amount of a compound of claim 1 and a pharmaceutically acceptable carrier or diluent.

14. A method for the treatment of disorders responsive to the inhibition of β-amyloid peptide production in a patient in need thereof, comprising administering a therapeutically effective amount of a compound of claim 1 to the patient.

15. The method of claim 14 wherein the disorder is Alzheimer's Disease or Down's Syndrome.
